SEQUENCE LISTING (32 sequences)

SEQ ID NO: 1 - DNA, 21 bp, artificial sequence
  oligonucleotide
ccgggtaccgagctcgaattc

SEQ ID NO: 2 - DNA, 36 bp, artificial sequence
  oligonucleotide
cgggatccggtggccagaactatggacttaatatac

SEQ ID NO: 3 - DNA, 30 bp, artificial sequence
  oligonucleotide
ccggaattcttaatttatcccatcaacgac

SEQ ID NO: 4 - DNA, 34 bp, artificial sequence
  oligonucleotide
cgggatccggtggtggtgacacaatattataggc

SEQ ID NO: 5 - DNA, 28 bp, artificial sequence
  oligonucleotide
ccggaattcttagatgcatattctgcac

SEQ ID NO: 6 - DNA, 34 bp, artificial sequence
  oligonucleotide
cgggatccggtggtggtcattcaattcaaactgg

SEQ ID NO: 7 - DNA, 27 bp, artificial sequence
  oligonucleotide
ccggaattcttacttgtcaatggtgaa

SEQ ID NO: 8 - DNA, 31 bp, artificial sequence
  oligonucleotide
ccggatccatgagatttccttcaatttttac

SEQ ID NO: 9 - DNA, 33 bp, artificial sequence
  oligonucleotide
gcgaattcagcacctcttttatccaaagatacc

SEQ ID NO: 10 - DNA, 31 bp, artificial sequence
  oligonucleotide
ccatcgatggttctgctagcgccaaaagctc

SEQ ID NO: 11 - DNA, 28 bp, artificial sequence
  oligonucleotide
cagctcgagttagaatagcaggtacgac

SEQ ID NO: 12 - DNA, 26 bp, artificial sequence
  oligonucleotide
aattcgacacaatatgtataggctac

SEQ ID NO: 13 - DNA, 22 bp, artificial sequence
  oligonucleotide
cgacacaatatgtataggctac

SEQ ID NO: 14 - DNA, 25 bp, artificial sequence
  oligonucleotide
accaagcttgatgcatattctgcac

SEQ ID NO: 15 - DNA, 28 bp, artificial sequence
  oligonucleotide
cggaattccattcaattcaaactggaag

SEQ ID NO: 16 - DNA, 27 bp, artificial sequence
  oligonucleotide
accaagcttcttgtcaatggtgaatgg

SEQ ID NO: 17 - DNA, 30 bp, artificial sequence
  oligonucleotide
cggaattccagaactatggacttaatatac

SEQ ID NO: 18 - DNA, 27 bp, artificial sequence
  oligonucleotide
accaagcttatttatcccatcaacgac

SEQ ID NO: 19 - DNA, 27 bp, artificial sequence
  oligonucleotide
cggaattcatggctagcaaaggagaag

SEQ ID NO: 20 - DNA, 26 bp, artificial sequence
  oligonucleotide
ggaagcttatcgatgttgtacagttc

SEQ ID NO: 21 - DNA, 39 bp, artificial sequence
  oligonucleotide
aattttacccatacgacgtcccagattacgctggtgccg

SEQ ID NO: 22 - DNA, 39 bp, artificial sequence
  oligonucleotide
aattcggcaccagcgtaaactgggacgtcgtatgggtaa

SEQ ID NO: 23 - DNA, 30 bp, artificial sequence
  oligonucleotide
aatttcatcaccatcaccatcacggtgccg

SEQ ID NO: 24 - DNA, 30 bp, artificial sequence
  oligonucleotide
aattcggcaccgtgatggtgatggtgatga

SEQ ID NO: 25 - DNA, 47 bp, artificial sequence
  oligonucleotide
taggcccagccggccgccgctagcaaaggagaagaactcttcactgg

SEQ ID NO: 26 - DNA, 31 bp, artificial sequence
  oligonucleotide
aaggcgcgccatcgatgttgtacagttcatc

SEQ ID NO: 27 - DNA, 40 bp, artificial sequence
  oligonucleotide
taggcccagccggccgcccagaactatggacttaatatac

SEQ ID NO: 28 - DNA, 28 bp, artificial sequence
  oligonucleotide
aaggcgcgccatttatcccatcaacgac

SEQ ID NO: 29 - DNA, 39 bp, artificial sequence
  oligonucleotide
taggcccagccggccgccgacacaatatgtataggctac

SEQ ID NO: 30 - DNA, 29 bp, artificial sequence
  oligonucleotide
aaggcgcgccgatgcatattctgcactgc

SEQ ID NO: 31 - DNA, 40 bp, artificial sequence
  oligonucleotide
taggcccagccggccgcccattcaattcaaactggaagtc

SEQ ID NO: 32 - DNA, 28 bp, artificial sequence
  oligonucleotide
aaggcgcgcccttgtcaatggtgaatgg

(19) United States
(12) Patent Application Publication
(10) Pub. No.: US 2005/0075298 A1
(43) Pub. Date: Apr. 7, 2005

(54) Methods and composition for delivering nucleic acids and/or proteins to the intestinal mucosa

(76) Inventors: Wei Chen, San Diego, CA (US); Xiaoli Fu, Carlsbad, CA (US); Sherry Nouraini, Vista, CA (US); Zhiqing Zhang, Carlsbad, CA (US)

Correspondence Address: ATTN: Louis C Cullman;Stradling Yocca Carlson & Rauth, Suite 1600, 660 Newport Center Drive, Newport Beach, CA, 92660, US

(21) Appl. No.: 10/353,149

(22) Filed: Jan. 27, 2003

Related U.S. Application Data
(63) Continuation-in-part of application No. 10/280,769, filed on Oct. 25, 2002.
(60) Provisional application No. 60/401,465, filed on Aug. 5, 2002.
(60) Provisional application No. 60/353,885, filed on Jan. 31, 2002.
(60) Provisional application No. 60/353,923, filed on Jan. 31, 2002.
(60) Provisional application No. 60/353,964, filed on Jan. 31, 2002.

(51) Int. Cl. A61K 48/00

(52) U.S. Cl. 514/44

(57) ABSTRACT

Methods and compositions are provided for in vivo heterologous nucleic acid delivery using genetically modified microflora. Specifically, compositions and related methods for the delivery of heterologous nucleic acids to the intestinal mucosa of animals are provided. Specifically, genetically modified microflora are used to deliver transforming heterologous nucleic acids directly, or genetically modified microflora expressing at least one heterologous nucleic acid are provided. Representative microflora include bacteria, bacterial fusions, and yeast. The heterologous nucleic acid may encode for immunoprotective epitopes (antigens) or other gene therapy applications.

### RELATED APPLICATIONS

This application claims priority to provisional application Ser. Nos. 60/401,465 filed Aug. 5, 2002, 60/353,885 filed Jan. 31, 2002, 60/353,923 filed Jan. 31, 2002, and 60/353,964 filed Jan. 31, 2002 and is a continuation-in-part of co-pending U.S. patent application Ser. No. 10/280,769 filed Oct. 25, 2002 the contents of which are incorporated herein by reference in their entirety.

### FIELD OF THE INVENTION

The present invention relates to the fields of bacteriology, immunology and gene therapy. Generally, the present invention pertains to in vivo heterologous nucleic acid delivery using genetically modified microflora. Specifically, the present invention includes compositions and related methods for heterologous nucleic acid delivery to the intestinal mucosa using genetically modified microflora to deliver transforming heterologous nucleic acids directly, or genetically modified microflora expressing at least one heterologous nucleic acid. The microflora of the present invention includes bacteria, bacterial fusions, and yeast. The heterologous nucleic acid may encode for immunoprotective epitopes (antigens) or other gene therapy applications.

### References

Various publications or patents are referred to in parentheses throughout this application to describe the state of the art to which the invention pertains. Each of these publications or patents is incorporated by reference herein. Complete citations of scientific publications are set forth in the text or at the end of the specification.

### BACKGROUND OF THE INVENTION

Diseases generally result from either defective or damaged genes or from exposure to infectious agents. Examples of diseases caused by defective genes include different forms of cancer such as colon and lung cancer, hemophilia, or low density lipid (LDL) receptor deficiency. Infectious diseases are generally caused by pathogenic microorganisms including bacteria, fungi, parasites, viruses and in some cases infectious defective proteins known as prions.

Modern gene therapy techniques have the potential to provide effective means to treat and/or prevent diseases cased by either defective genes or infectious agents. Gene therapy or gene replacement therapy involves providing a host with heterologous nucleic acids to replace defective genes or heterologous nucleic acids that encode for immunoprotective epitopes derived from infectious agents or tumors. In the later case the heterologous nucleic acid encoding an immunoprotective epitope can be used to either transform a host cell or be expressed on the surface of, and/or secreted from the gene delivery vehicle (vector).

Common vectors for introducing the heterologous nucleic acids include viral and non-viral vectors. Although viral delivery systems have been considered to be most efficient in delivering genes to cells, it may be limited because of a risk of triggering inflammatory or immunogenic responses to the delivery vector. Forbes, S. J., Review Article: Gene Therapy in Gastroenterology and Hepatology, Aliment Pharmacol. Ther. 11:823-826 (1997). Examples of viral vectors include retroviral vectors, adenoviral vectors, and adenoviral associated viral vectors. Although adenoviral vectors offer several advantages over other viral vectors in that they can infect a wide range of cells and are not limited to replicating cells, as are retroviral vectors, adenoviral vectors may activate the immune system such that the initial dose or repeated introduction may become less effective, if not life threatening. See also Forbes, S. J., supra.

Heterologous nucleic acids delivery may also be achieved using plasmid formulated with various transfecting agents. The most basic transfecting agent would be saline where plasmid DNA in saline is injected in muscles and has been shown to be delivered into the muscle cells. Other examples include liposomes, PEG, and various polymers. However, current modes of gene delivery and immunization techniques in vivo rely heavily on injection into the muscles, skin, vein, peritoneal, and other body sites.

Immunotherapeutics and prophylactic vaccines represent one of the most promising areas of disease prevention. Immunotherapeutics are genrally considered compositions that help sequester and kill pathogens or tumors and are administered after the disease process is established. Prophylactic vaccines are used to prevent infection of cancer and are administers prior to initiation of the disease process. However, both immunotherapeutics and vaccines must be formulated to elicit a specific immune response in order to be efficacious.

There are at least two immune systems, a “peripheral” or “systemic” and a “mucosal” immune system (Ogra et al., 1994). These systems operate both separately and simultaneously but may interact with one another via specific lymphocytic modulators to mount an effective immune response. The determining factor for which immune response will react first is the way in which pathological antigens are acquired by the individual and processed by the various lymphatic tissues.

Mounting an effective immune response depends upon the continuous movement of lymphocyte associated cells through blood, tissue and lymph (Anderson and Shaw, 1996). Lymphoid cells travel to the secondary lymphoid organs of the spleen, lymph nodes and to specialized mucosal tissue called Peyer's patches to encounter antigens acquired from the environment via blood, lymph or across mucous membranes, respectfully. Where and by which cells antigens are presented to these trafficking lymphatic cells significantly influences the outcome of the immune response with respect to T cell activation and B cell conversion into a particular antibody isotope and future homing preference of memory and effector lymphoid cells.

Antigens in lymph are filtered, trapped, processed and presented where the lymph passes over fixed antigen-presenting cells in lymph nodes. This antigen presentation by lymph nodes primarily results in “peripheral” immunity and the conversion of appropriate B cells into the specific IgG or IgM antibody. Antigens in blood are presented at specific blood/tissue interfaces in the spleen, which also primarily results in evoking “peripheral” immunity, however, due to the spleen's function of accommodating both antigen-presenting cells and activated T-and B cells from various other tissues, it is possible that cross talk between the two systems may amount to either peripheral or mucosal immunity or both. Antigens in the lumens of enteric organs (i.e., the respiratory and gastrointestinal tracts) are non-destructively endocytosed by specialized epithelial cells called “M” cells and transcytosed onto lymphoid cells in the Peyer's patches where response to antigen presentation primarily triggers commitment to “mucosal” immunity and the release of specific IgA antibodies into mucosal secretions.

The spaces inside the nose, throat, lungs and intestinal mucosa are continuous with the outside world, exposing these tissues to toxic. and pathogenic threats from the environment. For protection, the respiratory, gastrointestinal and urogenital tracts are composed of mucosal surfaces made of a layer of mucus coated epithelial cells, joined cell to cell by gasketlike intercellular tight junctions. Facing an environment rich in microflora, these mucosal surfaces present a cellular barrier that is the first interface between pathogens and host. Thus they are critical in the prevention of infectious diseases.

Though similar in purpose, the epithelial linings of the different mucosal surfaces in the body differ dramatically. Multilayered squamous epithelia line the oral cavity, pharynx, esophagus, urethra, and vagina while the mucosal surfaces of the gastrointestinal tracts are lined by a single layer of simple epithelial cells. Together these mucosal surfaces have a combined surface area of over 400 m². In the intestine, the epithelial cells of the small and large intestines are well equipped to face such a pathogen-rich foreign environment. Though this vast cellular barrier consists of a delicate monolayer of cells actively engaged in digestion and absorption of nutrients, it is generally able to exclude potentially harmful and antigenic materials.

Within this mucosal epithelial lining of the intestinal mucosa, bits of lymphoid tissue make up the organized mucosa lymphoid follicle-associated epithelium (FAE) tissue. Though the epithelium that lines the intestinal mucosa is impermeable to macromolecules and microorganisms, in mucosal inductive sites, such as the Peyers patches in the intestinal tract, the lymphoid FAE contains microfold, or M cells, that allow the transportation of antigens and microorganisms, for antigen sampling. M cells, in simple epithelia only occur over organized lymphoid follicles. Hence, at FAE sites, rich in M cells, there is a highly developed collaboration of the specialized epithelia with antigen-presenting and lymphoid cells. Through active transepithelial vesicular transport, M cells transport macromolecules, particles, and microorganisms for the lumen, across their cytoplasm and directly into the intraepithelial mucosal lymphoid follicles and to organized mucosal lymphoid tissues that are designed to process antigens and initiate a mucosal immune response that results in secretory immunity—the process by which mucosal surfaces of the intestinal mucosa and lung are bathed with protective antibodies.

Hence, M cells provide local, functional openings in the epithelial barrier through which vesicular transport occurs. Restriction of M cells to the sites directly over lymphoid follicles (FAE) serves to reduce the inherent risk of transporting foreign material and microbes across the epithelial barrier by assuring immediate exposure to phagocytes and antigen-presenting cells. The apical surfaces of M cells, facing the lumen, are distinguished from neighboring cells by the absence of a typical brush border and the presence of variable microvilli or microfolds with large intermicrovillar endocytic domains. A basal invagination in M cells creates a unique feature of the M cell, which is an intraepithelial “pocket” or space that both shortens the distance that transcytotic vesicles must travel from the apical to the basolateral surface and provides a docking site for lymphocytes, such as B and CD4 T cells, macrophages and dendritic cells to gather. M cells also have basal processes that extend into the underlying lymphoid tissue where they make direct contact with lymphoid and/or antigen-presenting cells, which likely plays a role in the presentation of antigens after M cell transport.

Parenteral immunization is the most common route of vaccination. It usually elicits a peripheral acute immune response, with protective IgM/IgG antibodies and peripheral cell-mediated immunity. The acute response soon abates, but it leaves behind sentries, known as “memory” cells, that remain on alert, ready to unleash whole armies of defenders if the real pathogen ever finds its way back into the body. Effective as they are, injected vaccines initially bypass mucous membranes and usually fail to stimulate mucosal lymphatic tissues to generate protective IgA antibodies and therefore they fail to stimulate mucosal immunity.

This presents a problem because many hazardous agents that spread through the systemic circulation initially infect across the mucosae, entering the body through the nose, mouth or other openings. Hence, the first defenses they encounter are those in the mucous membranes that line the airways, the digestive tract and the reproductive tract; these membranes constitute the biggest pathogen-deterring surface in the body. Protection against these agents requires vaccines that not only induce a peripheral but also a mucosal immune response. As stated above, when the mucosal immune response is initiated, it generates IgA antibodies that dash into the cavities of those passageways, neutralizing any pathogens they find. An effective reaction also activates a systemic response, in which circulating cells of the immune system help to destroy invaders at distant sites.

Another complication with respect to “paranteral” vaccination is that classic vaccines pose a risk that the vaccine microorganisms will somehow spring back to life, causing the diseases they were meant to forestall.

Because of this complication alternative approaches to traditional modes of vaccination are being sought. One of these is the use of DNA vaccines., wherein a plasmid containing a DNA segment from a pathogenic organism is administered to induce protection against various pathogens, including hepatitis B virus, herpes simplex virus, HIV, malaria and influenza.

The methods currently under development with respect to DNA vaccines are also plagued with problems. First of all, delivery is complicated. The gene or cDNA needs to be incorporated into an appropriate expression vector and delivered into an appropriate protein-synthesizing organism (e.g., E. coli, S. cerevisiae, P. pastoris, or other bacterial, yeast, insect, or mammalian cell) for the production of multiple copies of the gene of interest. Further, the DNA must be isolated, put into another expression system and delivered into a host, where the gene, under the control of an endogenous or exogenous promoter, can be appropriately transcribed and translated. The use of multiple expression vectors (including, but not limited to, phage, cosmid, viral, and plasmid vectors) are expensive, difficult to make, and hard to administer. Further, effective administration often requires the co-administration of viral elements for delivery into the host, which carries the risk of recombinant competent retrovirus formation.

Another method for inducing immunoprotection provides the administration of subunit vaccine preparations, composed primarily of antigenic proteins divorced from a pathogen's genes. By themselves, these proteins have no way of establishing an infection. However, induction of antibodies and CTL in the systemic but not the mucosal compartment normally results, further these vaccines are expensive to produce, purify and maintain.

A further problem related to traditional modes of vaccination is that physiological changes in the human host may be contributing to the emergence of new diseases. Perhaps emerging pathogens become resistant to antibiotics or (through genetic recombination) become more resistant to host defenses. Recombination events or lack of exposure can result in loss of immunity of the population to the pathogen, as has been well documented with influenza virus. Recombination events increase the infection rate by the emerging pathogen and, in the case of influenza virus, occasionally result in pandemics.

Hence, despite advances in disease prevention and immunization, new and reemerging infectious diseases are tipping the balance in favor of the parasite; systemic immunization is important but continued development of mucosal vaccines will be needed to effectively combat these new threats. For this reason, oral vaccines are currently being developed. They are better at evoking both a “mucosal” and a “peripheral” immune response, more cost effective and they are more convenient than vaccines of the more commonly used parenteral delivery system. Currently, the oral vaccines being developed tend to focus on the development and utilization of modified pathogenic organisms, such as Salmonella species, as antigen carriers for oral immunization (Stocker, U.S. Pat. No. 4,837,151, Auxotrophic Mutants of Several Strains of Salmonella; Clements et al., U.S. Pat. No. 5,079,165, Avirulent Strains of Salmonella; Charles et al., U.S. Pat. No. 5,547,664, Live-attenuated Salmonella). However, even when these pathogens are attenuated they may pose a danger of reverting to pathogenicity and being harmful to the host animal.

Given the problems inherent in parenteral vaccination, especially as they relate to DNA or sub-unit vaccines, the present inventor has developed novel compositions and methods of using non-pathogenic Lactic acid bacteria (LAB) as a live vehicle for the production and delivery of therapeutic molecules such as antigens. LAB, in general, and Lactobacillus species in particular, possess certain properties that make them attractive candidates for use in oral vaccination. These properties include adjuvant activity, mucosal adhesive properties, low intrinsic immunogenicity and they are regarded as safe (GRAS). They are already present in endogenous intestinal flora and are used commercially in the production of yogurts, cultured milks and other foods as well as for probiotic applications. Dietary LAB have been consumed for a long time and are generally recognized as safe, which represents an important advantage for their potential use as live therapeutic vehicles. One particular recombinant LAB currently being studied by the present inventor for use as live vaccine vehicle is Lactobacilli. The ubiquity of Lactobacillus species in the mammalian gastrointestinal tract combined with their ability to target and adhere to mucosal receptors make them useful organisms as vectors for vaccinating a host against a wide range of pathogens.

With respect to Lactic Acid Bacteria in general, several procedures already exist for the creation of LAB transformants. Leer et al. (WO095/35389) disclose a method for introducing nucleic acid into microorganisms, including microorganisms such as Lactobacillus and Bifidobacterium. The method of Leer et al. is based on limited autolysis before the transformation process is undertaken. Published PCT application PCT/NL96/00409 discloses methods for screening non-pathogenic bacteria, in particular LAB of the genera Lactobacillus and Bifidobacterium, for the ability to adhere to specific mucosal receptors. An expression vector is also disclosed that comprises an expression promoter sequence, a nucleic acid sequence, and sequences permitting ribosome recognition and translation capability. This reference indicates that various strains of Lactobacillus can be transformed so as to express heterologous gene products including proteins of pathogenic bacteria.

Further, oral administration of recombinant L. lactis has been used to elicit local IgA and/or serum IgG antibody responses to an expressed antigen. Wells et al, Mol. Microbiol. 8: 1155-1162,1993. In addition, Casas et al. (U.S. Pat. No. 6,100,388) discloses that L. reuteri, can be transformed with heterologous DNA, and can express the foreign protein on the cell surface or secrete it, while EP 1084709 A1 discloses that L. plantarum can, as well, be transformed to express an antigenic fragment either intracellularly or on the cell surface. See also See U.S. Pat. Nos. 5,149,532 and 6,100,388.

These references all disclose the use of certain species of bacteria for use in vaccination. The methods therein described are altogether time consuming, expensive and inefficient. In addition, with respect to the methods currently practiced, different expression systems can be required for each specific species sought to be used for antigen delivery. Appropriate promoters, enhancers and selectable markers often have to be developed. Several different transformations may need to take place to determine a viable system so as to ensure appropriate expression levels in vitro and in vivo. All of this adds both tremendous time and cost. What is needed is a system whereby previously known and commercially available expression systems may be used to express heterologous protein elements in commercially available, safe, food grade microflora for the purposes of both disease prevention and treatment.

### SUMMARY OF THE INVENTION

The present invention generally pertains to in vivo heterologous nucleic acid delivery using genetically modified microflora. Specifically, the present invention includes compositions and related methods for heterologous nucleic acid delivery using genetically modified microflora to deliver transforming heterologous nucleic acids directly, or genetically modified microflora expressing at least one heterologous nucleic acid. The genetically modified microflora of the present invention include bacteria, bacterial fusions, and yeast. The heterologous nucleic acid may encode for immunoprotective epitopes (antigens) or other proteins suitable for gene therapy applications including gene replacement and/or gene augmentation.

In one embodiment of the present invention an immunogenic composition is provided comprising genetically modified microflora expressing at least one heterologous nucleic acid. The genetically modified microflora include, but are not limited to bacteria, bacterial fusants, yeast and yeast-bacteria fusants. The heterologous nucleic acid may encode for immunoprotective epitopes (antigens) that may be either prophylactic or therapeutic or both.

In another embodiment of the present invention the antigens are expressed on the surface of the genetically modified.

In yet another embodiment of the present invention the antigen is n is both secreted and expressed on the surface of the genetically modified microflora. When the antigen expressing genetically modified microflora of the present invention come into contact with immune cells of the intestinal mucosa an immune response directed against the antigen is elicited.

In still another embodiment the present invention provides compositions and related methods for using bacteria and yeast from the natural flora, or “microflora” of the human body to deliver DNA to mammalian cells. In one embodiment of the present invention a culture of modified microflora bacteria carrying a DNA vector such as a plasmid with a mammalian expression system may be introduced into the intestines through oral ingestion of the culture.

When the compositions of the present invention are administered orally, intestinal cells such as, but not limited to, enterocytes, M-cells, K-cells and any other cells lining the intestines of the body or cells underneath the layer of epithelial cells are targeted. In one embodiment, genetically modified microfloral carries an autolysing gene operably linked to an inducible promoter that can be induced, for example, by pH drop, lactose, temperature, anaerobic condition or any other suitable inducer or inducing conditions. When the autolysing protein acts upon the bacterial cells, the bacterial cells may then rupture and release the plasmid inside the gastrointestinal tract, preferably in the intestinal lumen. The plasmid preferably is a high copy number plasmid such as pUC18-based plasmid or a “runaway” plasmid.

In another embodiment, genetically modified microflora organisms of the present invention may be provided as protoplasts, i.e., bacterial cells without their cell wall. As the protoplasts travel down the gastrointestinal tract, the hypotonic environment would create osmotic pressure within the bacterial cells to burst the cells and release the plasmid.

In yet another embodiment, the genetically modified microflora of the present invention may be treated with lytic virues including, but not limited to bacteriophages such as φadh, φLC3, mv4, M13, T4, φ29, Cp-1, Cp-7, and Cp-9 bacteriophages before ingestion. As the microflora cells travel down the gastrointestinal tract, the bacteriophages may undergo their lytic cycle and lyse the microflora cells releasing the plasmid.

Because of the sheer volume of bacteria and the large amount of DNA released, the intestinal cells in the body will take up the DNA into the cells and express the proteins. Uptake may occur due to the association of the plasmid, for example, with cell membrane debris, which may act as carriers just as liposomes act as carriers of DNA in liposomal transfection. The intestinal cells may also take up naked DNA by endocytosis or pinocytosis.

Furthermore, because the M-cells in the intestinal cells are normally involved in vesicular transport of macromolecules, the DNA released may be taken up by the M-cells. As M-cells are situated in lymphoid follicles associated epithelium (FAE) which has high number of immune cells such as B and T cells, mucosal and/or peripheral immune response to the protein expressed by the plasmid may be expected. With respect to delivering therapeutic genes such as insulin, interferon, growth hormone, factor VIII and IX or any other desired therapeutic protein, M-cell pockets may be a gateway for secreted therapeutic proteins to enter the blood stream. In one embodiment, DNA coding region of proteins may be genetically engineered or spliced downstream of a signal sequence peptide that allows for secretion of the protein.

The genetically modified microflora organisms of the present invention may be provided, for example, as dry powder reconstituted before ingestion or enclosed in capsules, or mixed into foods such as milk, yogurt, or ice cream.

### BRIEF DESCRIPTION OF THE FIGURES

FIG. 1 depicts the expression of Green Fluorescent protein (GFP) on the surface of yeast cells transformed in accordance with the teachings of the present invention.

FIG. 2 graphically depicts the serological results from mice receiving an oral vaccine against influenza virus using the GPD plasmid versus controls.

FIG. 3 graphically depicts the serological results from mice receiving a subcutaneous vaccine influenza virus using the GPD plasmid versus controls.

FIG. 4 graphically depicts the serological results from mice receiving an oral vaccine against rotavirus VP7 using the GPD plasmid versus controls.

FIG. 5 graphically depicts the serological results from mice receiving a subcutaneous vaccine against rotavirus VP7 using the GPD plasmid versus controls.

FIG. 6 graphically depicts the serological results from mice receiving an oral vaccine against influenza virus using the pYD plasmid versus controls.

FIG. 7 graphically depicts the serological results from mice receiving a subcutaneous vaccine against influenza virus using the pYD plasmid versus controls.

FIG. 8 graphically depicts the serological results from mice receiving an oral vaccine against rotavirus VP7 using the pYD plasmid versus controls.

FIG. 9 graphically depicts the serological results from mice receiving a subcutaneous vaccine against rotavirus VP7 using the pYD plasmid versus controls.

FIG. 10 graphically depicts the serological results from mice receiving an intranasal vaccine against influenza virus using the pYD plasmid versus controls.

### DEFINITIONS

Various terms relating to the biological molecules of the present invention are used throughout the specification and claims. Prior to setting forth the invention, it may be helpful to an understanding thereof to setforth definitions of the terms that will be used hereinafter.

“Antigen” or “antigenic fragment,” immunoprotective epitope” or “epitope” refers to all or parts thereof of a protein or peptide capable of causing a cellular or humoral immune response in a subject (i.e., an animal or mammal). Such would also be reactive with antibodies from animals immunized with said protein. Furthermore, the terms “antigen,” “antigenic fragment” or “epitope” as used herein describing this invention, include any determinant responsible for the specific interaction with an antibody molecule. Antigenic or epitopic determinants usually consist of chemically active surface groupings of molecules such as amino acids or sugar side chains and have specific three-dimensional structural characteristics, as well as specific charge characteristics. Examples of antigens or epitopes that can be used in this invention include, but are not limited to, viral, bacterial, protozoan, microbial and tumor antigens.

An “antigenic or therapeutic element” may include, for example, antigenic or therapeutic DNA, cDNA, RNA, and antisense polynucleotide sequences.

A “coding sequence” or “coding region” refers to a nucleic acid molecule having sequence information necessary to produce a gene product, when the sequence is expressed.

The term “compatible” with reference to a mammalian body refers to the capability of co-existence, together in harmony, i.e., capable of being used in transfusion or grafting without immunological reaction.

The term “contacted” when applied to a cell is used herein to describe the process by which an antigen or therapeutic gene, protein or antisense sequence, and/or an accessory element, is delivered to a target cell, via a microflora delivery vehicle, or is placed in direct proximity with the target cell.

“Delivery of a therapeutic agent” may be carved out through a variety of means, such as by using oral delivery methods such as pill formulations or compositions formulated in such a way as to allow for oral administration, and the like. Such methods are known to those of skill in the art of drug delivery, however, preferable compositions include pharmaceutical formulations, comprising a antigenic or therapeutic gene, protein, or antisense polynucleotide sequence that may be delivered in combination with a microflora delivery vehicle, such as Lactobacillus or Saccharomyces. In such compositions, the gene may be in the form a DNA segment, plasmid, cosmid or recombinant vector that is capable of expressing the desired protein in a cell; specifically, a LAB-E. coli fusant cell. These compositions may be formulated for in vivo administration by dispersion in a pharmacologically acceptable grade of yogurt.

The term “expression cassette” refers to a nucleotide sequence that contains at least one coding sequence along with sequence elements that direct the initiation and termination of transcription. An expression cassette may include additional sequences, including, but not limited to promoters, enhancers, and sequences involved in post-transcriptional or post-translational processes.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. With respect to a protein, the term “heterologous” is herein understood to mean a protein at least a portion of which is not normally encoded within the chromosomal DNA of a given host cell. Examples of heterologous proteins include hybrid or fusion proteins comprising a bacterial portion and a eukaryotic portion, eukaryotic proteins being produced in prokaryotic hosts, and the like.

A “heterologous nucleic acid” is a DNA, cDNA or any form of RNA polynucleotide sequence, or hybrid thereof, as well as an amino acid sequence constituting a polypeptide, peptide fragment, or protein that is derived from a different species from the one in which it is being produced. Heterologous nucleic acid sequence may also include a nucleic acid sequence from the same species that is intended to replace or augment and endogenous nucleic acid sequence. This particularly true for gene therapy applications including gene replacement.

An “immunogenic composition” as used herein is an embodiment of the present invention that provides an antigen to an animal in a manner that facilitates the induction of an immune response. The immune response can be humoral or cellular or both and contains and immunogen, or a fragment or subunit thereos. Representative antigens include, but are not limited tumor antigens, viral antigens, parasitic antigens, fungal antigen and bacterial antigens. For example, bacterial antigens that may be encoded may include, but not hereby limited to, Mycobacterium leprae antigens; Mycobacterium tuberculosis antigens; Rickettsia antigens; Chlamydia antigens; Coxiella antigens; malaria sporozoite and merozoite proteins, such as the circumsporozoite protein from Plasmodium berghei sporozoites; diphtheria toxoids; tetanus toxoids; Clostridium antigens; Leishmania antigens; Salmonella antigens; E. coli antigens; Listeria antigens; Borrelia antigens, including the OspA and OspB antigens; Franciscella antigens; Yersinia antigens; Mycobacterium africanum antigens; Mycobacterium intracellular antigens; Mycrobacterium avium antigens; Shigella antigens; Neisseria antigens; Staphylococcus, Helicobacter, peudomona, Treponema antigens; Schistosome antigens; Filaria antigens; Pertussis antigens; Staphylococcus antigens; Anthrax toxin, Pertussis toxin, Clostridium; Hemophilus antigens; Salmonella; Streptococcus antigens, including the M protein of S. pyogenes and pneumococcus antigens such as Streptococcus pneumoniae antigens.

Viral antigens that may be encoded may include, but not hereby limited to, mumps virus antigens; hepatitis virus a.b.c.d.e. HBV antigens; rabies antigens; polio virus antigens; Rift Valley Fever virus antigens; dengue virus antigens; measles virus antigens; rotavirus antigens; Human Immunodeficiency Virus (HIV) antigens, including the gag, pol, and env proteins as well as gp120 and gp 160 of the HIV env; respiratory syncytial virus (RSV) antigens; Herpes virus antigens; parainfluenza virus antigens; measles virus antigens; snake venom antigens; human tumor antigens; Vibrio cholera antigens, as well as antigens from HCV, HAV, HPV, TB, Herpes, rubella, influenza, mumps, poliomyelitis, rotavirus, surface glycoprotein of malaria parasite, parvovirus, Epstein barr virus, poxvirus, rabies virus, pneumonia, cancer antigens like CEA and other similar antigenic fragments. fragment.

“Lactic Acid Bacteria” or “LAB” generally refers to a family of Gram positive bacteria that ferment carbohydrates to produce lactic acid as a final product. Lactic acid bacteria live in the oral cavities and the alimentary tract and are utilized for the manufacture of fermentative foods, such as kimchi, yogurt, etc. They are known to produce various antimicrobial compounds, such as organic acids, hydrogen peroxide, diacetyl and bacteriocins, and are known to play an important role in maintaining the entrails healthy condition by utilizing carbohydrates as an energy source to produce lactic acid and antibacterial materials which inhibit the growth of the harmful bacteria. Among the lactic bacteria are those of the genera Streptococcus, Enterococcus, Lactococcus, Lactobacillus, and Bifidobacterium. Representative examples of these lactic acid-producing bacteria include Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

“Lactobacillus” refers to a lactic acid bacteria of the genus Lactobacillus that has the following bacteriological properties: namely Gram positive, rod shape, non-mobility, negative catalase, facultative anaerobic properties, optimum growth temperature of 30° to 40° C., no growth at 15° C. and formation of DL-lactic acid.

“Microflora” as used herein includes bacterial, yeast, bacteria-bacteria fusants and bacteria-yeast fusants.

The term “modified” refers generally to a process whereby basic or fundamental changes are made to a given organism or system to bring about a new orientation or formation to or to serve a new end. In one embodiment a “modified microflora organism” is one that has been transformed with an expression vector encoding for an antigenic or therapeutic polypeptide and wherein the “modified microflora” expresses the antigenic or therapeutic polypeptide either on its surface and/or secretes it.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene. The term “DNA construct” is also used to refer to a heterologous region, particularly one constructed for use in transformation of a cell.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

A “plasmid” or a “plasmid vector” is a circular DNA molecule that can be introduced or transfected into bacterial or yeast cells by transformation, which plasmid will then replicate autonomously in the cell. A plasmid vector usually comprises a promoter sequence that is recognized by an RNA polymerase that may or may not be inherent to the host, which controls the expression of the desired gene, a heterologous nucleic acid operably linked to the promoter sequence, and a replication origin for increasing the copy number by induction with an exogenous factor. Plasmid replication origins are important because they determine plasmid copy number, which affects production yields. Plasmids that replicate to higher copy number can increase plasmid yield from a given volume of culture. (Suzuki et al., Genetic Analysis, p. 404, (1989). The promoter sequence contained in the plasmid vector, which sequence controls the expression of the desired gene, may be any promoter sequence capable of driving expression of the gene in that given host; i.e., promoter sequences recognized by particular RNA polymerases, e.g., those recognized by RNA polymerases derived from the T7, T3, SP6 and others such as LacZ, can be used. Promoters usable for this purpose include, but are not limited to, the lac, tip, tac, gal, ara and P.sub.L promoters etc. when Escherichia coli, is used so long as the above-described purpose is accomplished (Fitzwater, et al., Embo J. 7:3289-3297 (1988); Uhlin, et al., Mol. Gen. Genet. 165:167-179 (1979)). Furthermore, the plasmid vector may have a drug resistance gene used as a selection marker.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA or RNA, DNA or RNA that is a mixture of single- and double-stranded regions as well as hybrid molecules comprising a mixture of the above. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the gene-encoded amino acids. “Polypeptides” include amino acid sequences modified either by natural processes, such as posttranslational processing, or by chemical modification techniques that are well known in the art. Such modifications are well described in basic texts and in more detailed monographs; as well as in a voluminous research literature.

Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “reporter gene” refers to a gene that encodes a product that is detectable by standard methods, either directly or indirectly.

“Saccharomyces” generally refers to a yeast strain of the genus Saccharomyces cerevisiae, bakers yeast, is a unicellular microorganism that can exist as haploid or diploid forms, and reproduces by budding of daughter cells. Due to the ease of genetic manipulation of the S. cerevisiae genome, it has been extremely valuable in research efforts aimed at understanding basic biological phenomenon in eukaryotes. The genome of yeast has been completely sequenced and there is a wealth of information available with regards to the biology, genetics and molecular biology of this organism. In addition, well known and characterized tools for constitutive and inducible expression of heterologous proteins in yeast are available, which has made yeast a valuable tool for expression and purification of a host of therapeutic recombinant proteins. Furthermore, Saccharomyces yeast are widely used in the preparation of baked goods and vitamins, and in fermentation of alcoholic bevearages that are consumed by humans, which forms the basis of endowing yeast with the label of Generally Regarded As Safe (GRAS) for human consumption by the Food and Drug Administration.

In addition to being widely used in food and beverage preparation, yeast is part of the natural microflora resident in the human body. Resident strains of Saccharomyces cerevisiae have been isolated in healthy individuals from mucosal surfaces of the mouth and rectum. (See: Xu, J., C. M. Boyd, E. Livingston, W. Meyer, J. F. Madden, and T. G. Mitchell. 1999. Species and genotypic diversities and similarities of pathogenic yeasts colonizing women. J. Clin.Microbiol. 37:3835-3843.)

Unlike the opportunistic microfloral yeast species, such as Candida albicans, which can lead to fatal infections in immuncompromised patients, resident Saccharomyces cerevisiae are rarely associated with such devastating health effects. In addition, it has been shown that administration of live yeast to healthy individuals and animal models does not lead to colonization and pathogenicity (See: Maejima, K., K. Shimoda, C. Morita, T. Fujiwara, and T. Kitamura. 1980. Colonization and pathogenicity of Saccharomyces cerevisiae, MC16, in mice and cynomolgus monkeys after oral and intravenous administration Jpn.J Med.Sci.Biol. 33:271-276. See also Pecquet, S., D. Guillaumin, C. Tancrede, and A. Andremont. 1991. Kinetics of Saccharomyces cerevisiae elimination from the intestines of human volunteers and effect of this yeast on resistance to microbial colonization in gnotobiotic mice. Appl.Environ.Microbiol. 57:3049-3051. Non-limiting examples of Saccharomyces speicies sutiable for use in accordance with the teachings of the present invention include the group consisting of Saccharomyces cerevisiae, S. exiquus, S. telluris, S. dairensis., S. servazzii, S. unisporus, and S. kluyveri.

The term “selectable marker gene” refers to a gene encoding a product that, when expressed, confers a selectable phenotype such as antibiotic resistance on a transformed cell.

With respect to “therapeutically effective amount” is an amount of the polynucleotide, antisense polynucleotide or protein, or fragment thereof, that when administered to a subject along with the bacterial fusant carrier, is effective to bring about a desired effect (e.g., an increase or decrease in a M-cell mediated immune response) within the subject.

“Transcriptional and translational” control sequences are DNA regulatory sequences, such as promoters, enhancers, polyadenylation signals, terminators, and the like, that provide for the expression of a coding sequence in a host cell.

A number of methods for delivering therapeutic formulations, including DNA expression constructs, into cells (e.g., E. coli cells) are known to those skilled in the art. A cell has been “transformed” or “transfected” or “transduced” by an exogenous or heterologous DNA or gene when such DNA has been introduced inside the cell. The transforming DNA may or may not be integrated (covalently linked) into the genome of the cell. In prokaryotes, bacteria and yeast cells for example, the transforming DNA may be maintained on an episomal element such as a plasmid or ligated into host DNA at specific restriction sites. As used herein, the term “transduction,” is used to describe the delivery of DNA to a cell using viral mediated delivery systems, such as, adenoviral, AAV, retroviral, or plasmid delivery gene transfer methods. As used herein the term, “transfection” is used to describe the delivery and introduction of a genetic element to a cell using non-viral mediated means, these methods include, e.g., calcium phosphate- or dextran sulfate-mediated transfection; electroporation; glass projectile targeting; and the like. These methods are known to those of skill in the art, with the exact compositions and execution being apparent in light of the present disclosure.

A “vector” is a replicon, such as plasmid, phage, or cosmid to which another nucleic acid segment may be operably inserted so as to bring about the replication or expression of the segment.

### DETAILED DESCRIPTION

Microflora concentrations at different sites of the body vary greatly. For example, the mucous membrane of the mouth and the surface of teeth have high concentrations of bacteria, which pass along with saliva and chewed food into the esophagus and thereafter into the stomach, where the food is mixed with gastric juices and fluidized. The acidity of the gastric juice effectively destroys most of the bacteria that come into contact with it. Food stays in the stomach for around four hours and is gradually released into the small intestine. The proximal part of the small intestine is also acidic due to the acid entering from the stomach. In addition, bile acids secreted into the proximal part of the small intestine destroy bacteria, so the bacteria level is relatively low. As acidity decreases and the bile acids are diluted, the bacteria level in the terminal part of the small intestine increases. The small intestine, several meters long, is densely proliferated with microvilli, which increase the internal surface area of the mucous membrane so much so that the small intestine would cover the area of a tennis court if it were spread out. The large surface area enables the efficient breakdown of food and the subsequent absorption of nutrients through the mucous membrane into the blood stream. Most of the system's immunological tissue is in connection with the small intestine and can be found immediately under the epithelial cells of the mucous membrane.

Lactic Acid Bacteria (“LAB”) and yeasts possess certain properties that make them attractive candidates for use in delivery of DNA into the intestinal cells. They are already present endogenously in the intestinal flora and are used commercially in the production of yogurts, cultured milks and other foods as well as for probiotic applications and are generally regarded as safe (GRAS). They also have mucosal adhesive properties and low intrinsic immunogenicity. The ubiquity of LAB and yeast in the mammalian gastrointestinal tract combined with their ability to target and adhere to mucosal receptors make them useful organisms as vectors for delivery of DNA and antigens into the intestinal cells.

LAB can also be found in the stomach and in the proximal part of the small intestine because LAB tolerate acidity relatively well. This makes them an ideal vector for DNA and antigen deleivery through the stomach and into the intestines. There are five major genera of lactic acid bacilli including Streptococcus, Enterococcus, Lactococcus, Lactobacillus, and Bifidobacterium. Representative examples of these lactic acid-producing bacteria include Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei, Lactobacillus plantarum, L. gasseri, L. jenseni, L. crispatus, L. paracasei, L. rhamnosus, L. agilis, L. salivarius, L. pseudoplantarum, L. buchneri and L. reuteri. Lactobacillus rhamnosus GG (ATCC 53103), or more briefly Lactobacillus GG or LGG, for example, is a probiotic strain that has been isolated from healthy human intestinal flora. The probiotic effects of lactobacilli on human well being have been widely researched and documented in scientific journals. All of these genera and species may be used as vector for DNA delivery as according to one aspect of the present invention.

E. coli is another species of bacteria that is naturally occurring in the intestinal tract of mammals. For example, E. coli K-cells are responsible for producing vitamin K needed by the body for blood clotting. Because the E. coli bacteria has been extensively studied and used as a workhorse for modern genetic engineering, methods and techniques of modifying and manipulating E. coli bacteria are well known in the art. As such, modified E. coli may also be used as a vector for delivering DNA into intestinal cells according to one aspect of the present invention.

Yeasts are eukaryotic fungi that are commonly found in foods and as such often colonize portions of the human intestines. Yeasts such as those of the genus Saccharomyces are generally regarded as safe and therefore are ideal candidiates to be used in accordance with the teachings of the present invention. The genus Saccharomyces includes the species S. exiquus, S. telluris, S. dairensis, S. servazzii, S. unisporus, S. cerevisiae and S. kluyveri. S. cerevisiae strains are used as modern bakers' yeast and S. kluyveri has been found in traditional leavening agents, therefore these species are ideally suited for modification in accordance with the teachings of the present invention.

For the purpose of this disclosure, all naturally occurring bacteria and yeast found in the mammalian body will be referred to as the “microflora.”

According to one aspect of the present invention, microflora including bacteria such as LAB, E. coli and yeasts may be transformed with a plasmid DNA that comprises a mammalian expression system capable of expressing proteins of interest. In one embodiment of the present invention the bacteria used is Lactobacillus acidophilus. In another embodiment the microflora is Saccharomyces sp. Cultures of modified microflora may then be administered to an animal or human through oral ingestion. As the microflora travel through the gastrointestinal tract, the microflora cells are triggered to lyse and release the plasmid in the intestinal tract. By administrating a high amount of microflora coupled with the use of high copy plasmids, the plasmid DNA would, thus, be taken up by the intestinal cells and expressed inside the cells.

Transforming of E. coli with plasmids is well known in the art and requires no further explanation. Transformation of LAB may be performed using a limited autolysis method as described in Leer et al. (WO095/35389), which is hereby incorporated by reference in its entirety. Transformation may also be performed on various LAB of interest according to methods and techniques disclosed in the following references, which are hereby incorporated by reference as if fully set forth herein. Published PCT application PCT/NL96/00409 discloses methods for screening non-pathogenic bacteria, in particular LAB of the genera Lactobacillus and Bifidobacterium, for the ability to adhere to specific mucosal receptors. An expression vector is also disclosed that comprises an expression promoter sequence, a nucleic acid sequence, and sequences permitting ribosome recognition and translation capability. This reference indicates that various strains of Lactobacillus can be transformed so as to express heterologous gene products including proteins of pathogenic bacteria. PCT/NL95/00135 describes a multicopy expression vector for use in Lactobacillus with a 5′non-translated nucleic acid sequence comprising at least the minimal sequence required for ribosome recognition and RNA stabilization, followed by a translation initiation codon. Further, oral administration of recombinant L. lactis has been used to elicit local IgA and/or serum IgG antibody responses to an expressed antigen (Wells et al., Antonie van Leeuwenhoek 1996 70:317-330). In addition, Casas et al. discloses in U.S. Pat. No. 6,100,388 that L. reuteri, can be transformed with heterologous DNA, and can express the foreign protein on the cell surface or secrete it, while EP 1084709 A1 discloses the that Lactobacillus plantarum can, as well, be transformed to express an antigenic fragment either intracellularly or on the cell surface.

Methods for yeast transformation are also well known in that art. See for example co-pending U.S. patent application Ser. No. 10/280,769 filed Oct. 25, 2002 for additional details. See also “Guide to yeast genetics and molecular and cell biology” (2002) Edited by Christine Guthrie and Gerald Fink. These are two books in the Methods in Enzymology series. Volumes 350 and 351. Published by Academic Press and are herein incorporated by reference in their entirety.

In addition to the vectors disclosed in the above references, other plasmids suitable for LAB include, for example, pFXL03, pWV01, pGKV210 and pVA838. Some plasmids from Lactobacillus and Lactococcus may also be obtained from DSMZ, Braunschweig, Germany. Others have been described in the literature. For example, plasmid vectors suitable for Lactobacillus casei are described in many references, including Maassen, C., et al., “Instruments for oral disease-intervention strategies: recombinant Lactobacillus casei expressing tetanus toxin fragment C for vaccination or myelin proteins for oral tolerance induction in multiple sclerosis” Vaccine 17(17): 2117-28 (1999). In addition, plasmid vectors suitable for Lactobacillus plantarum and Lactococcus lactis are described in Geoffrey, M., et al., “Use of green fluorescent protein to tag lactic acid bacterium strains under development as live vaccine vectors” Applied and Environmental Microbiology 66(1): 383 (2000)). Plasmid vectors for Lactococcus lactis, Lactobacillus fermentum, and Lactobacillus sake are described in Piard, J., et al., “Cell wall anchoring of the Streptococcus pyogenes M6 protein in various lactic acid bacteria” Journal of Bacteriology 179(9): 3068-72 (1997). Some plasmid vectors are suitable for a wide range of Lactobacillus species, such as pPSC20 and pPSC22, described in Cocconcelli, P., et al., “Single-stranded DNA plasmid, vector construction and cloning of Bacillus stearothermophilus alpha-amylase in Lactobacillus” Res Microbiol 142(6): 643-52 (1991). Shuttle vectors, which are plasmids that are capable of expression in both of the Lactobacillus and E. coli could also be used. Lactobacillus/E. coli shuttle vectors are described in Maassen, C., et al., Vaccine, ibid. Because the methods and techniques for manipulating E. coli are so well characterized, shuttle vectors have the advantage of ease of working the genetic modification in E. coli and later transferring to lactobacillus. Vectors and plasmids for E. coli are also well known and abundantly available from many sources including commercial sources.

The plasmid may contain either selectable marker genes or reporter genes used to facilitate determining which bacteria contain the desired plasmid DNA. Possible selectable marker genes are antibiotic resistance markers such as kan^{r}, tetr and ampr, or enzyme mutation or deficiency that affects the bacteria's ability to metabolize or synthesize certain nutrients. The gene for Beta galactosidase and the gene encoding green fluorescent protein (GFP) are examples of reporter genes. Alternatively, if the plasmid does not include a selectable marker or reporter gene the plasmid DNA could be detected in a variety of ways, such as a dot blot using the plasmid DNA as a probe.

If desired, promoters for prokaryotic expression in LAB and E. coli bacteria may also be used and these have been described in the literature. For example, one promoter that is well suited for both Lactobacillus plantarum and Lactococcus lactis and that has also been shown to be useful for expression in other LAB is the nisin inducible nisA promoter from Lactococcus lactis. (deRuyter, P., et al., “Controlled gene expression systems for Lactococcus lactis with the food-grade inducer nisin” Appl. Environ. Microbiol. 62: 3662-67 (1996)) Kleerebezem, M., “Controlled gene expression systems for lactic acid bacteria: transferable nisin-inducible expression cassettes for Lactococcus, Leuconostoc, and Lactobacillus spp.” Applied and Environmental Microbiology 63(11): 4581-84 (1997)) The L. plantarum IdhL promoter has also been used successfully in L. plantarum. Promoters for L. casei expression systems include the constitutive lactate dehydrogenase promoter from L. casei and the regulatable amylase promoter from L. amylovorus (Maassen, C., et al., Vaccine 17(17): 2117-28 (1999)). The lactococcal promoter P₅₉ has been used in expression vectors of various Lactococcus lactis and Lactobacillus bacteria (Piard, J., et al., “Cell wall anchoring of the Streptococcus pyogenes M6 protein in various lactic acid bacteria” Journal of Bacteriology 179(9): 3068-72 (1997)). In addition, the plasmids could contain multiple promoter sequences all operably linked to the sequence encoding the antigen.

In a one embodiment of the invention, the DNA construct will be a plasmid encoding at least an appropriate origin of replication for the desired bacterial host, a selectable marker gene and/or a reporter gene, a promoter operably linked to a heterologous nucleotide sequence encoding the antigen or therapeutic element fused to surface binding or secretion signal sequence. The construct may also contain other suitable elements, such as transcription initiation sequences, anchor or secretion signal sequences and transcription termination sequences.

Vectors for used with LAB may include modifications of existing LAB plasmids disclosed in the above mentioned references. In particular, through recombinant DNA techniques, the plasmid vector may be modified to include an eukaryotic expression enhancers and promoters such as CMV promoter, RSV promoter, ubiquitin promoter, actin promoter, or any other suitable promoter capable of expressing the protein in a mammalian cell. The modification may also include the introduction of 5′ untranslated region in the promoter such as Intron A of the CMV promoter to enhance expression in mammalian cells. Furthermore, 3′ untranslated regions comprising the poly A tail and polyadenylation signals may also be included into the plasmid for proper processing of mRNA molecules expressed from the plasmid. In certain instances where expression of protein in LAB is not required, the prokaryotic expression system may be deleted except for the origin of replication sequences and selection markers.

The modified plasmid for eukaryotic expression may then be used to express any protein of interests. Examples include reporter proteins (such as Green Fluorescent Protein (GFP), luciferase, secreted alkaline phosphastase, CAT, etc.), therapeutic proteins (such as insulin, growth hormone, interferon, erythropoietin, filgastrim, cytokines, interleukins, human albumin, activase, enzymes for vitamins synthesis or lactose digestion (lactase), Factor Vil and IX, whole antibodies, antibody fragments, antibiotics, hormones, pheromones, other small molecules like calcitonin), and antigenic proteins (such as bacterial toxins, viral proteins, or cancer proteins). To increase the immunogenic response of the body to the antigenic proteins, the antigen may be co-expressed with Interleukin-2 either in separate vectors, same vector different expression cassette, or through an IRES sequence for bicistronic expression. See, e.g., Chow, Y. H., et al., (1997) “Improvement of Hepatitis B Virus DNA Vaccines by Plasmids Coexpressing Hepatitis B Surface Antigen and Interleukin-2,” J. Virology, Vol. 71, No. I, 169-178.

For secretion of the translated protein into the extra-cellular environment, appropriate secretion signals may be incorporated into the desired polypeptide. These signals may be endogenous to the polypeptide or they may be heterologous signals. Hence, secretion signals may be used to facilitate delivery of the resulting protein. The coding sequence for the secretion peptide is operably linked to the 5′ end of the coding sequence for the protein, and this hybrid nucleic acid molecule is inserted into a chosen plasmid adapted to express the protein in the host cell of choice. Plasmids specifically designed to express and secrete foreign proteins are available from commercial sources such as pSecTag2 vectors from Invitrogen Corporation. Because intestinal cells are bipolar, appropriate signal sequence for apical or basolateral targeting may also be used.

Furthermore, the plasmid vector may be modified by recombinant DNA techniques to include a viral origin of replication such as the oriP/EBNA-1 protein from Epstein-Barr virus. Huertas, D., et al., “Expression of the human CFTR gene from episomal oriP-EBNAI-YACs in mouse cells,” Human Molecular Genetics, 2000, Vol. 9, No. 4, 617-629. With a viral origin of replication, the plasmid can be maintained as stable episomes in mammalian cells because it replicates with the endogenous DNA and segregates by sticking to the host chromosomes. Thus, the plasmid may be maintained for longer period of time resulting in long term expression of a particular protein.

In one embodiment of the present invention, modified microflora, as described above, may be prepared by treating the bacteria with lysozymes to form protoplasts as exemplified in Example I, discussed below. Cultures of protoplasts may then be orally ingested. As the protoplasts passes through the stomach and into the intestines, osmotic pressure and/or bile salts will trigger the bursting of the protoplasts to release the plasmid DNA. To increase the chances that the protoplasts to withstand the gastric acids of the stomach, the protoplasts may be formulated with various carriers and buffers to protect against the acids in the stomach. For example, NaHCO3, often provided in antacids, may be used as a buffer to formulate the protoplasts in isotonic solution for oral ingestion. Other examples include formulating the protoplasts in gels such as agarose, gelatins, etc.

In another embodiment, the lysis occurs through the infection of the bacterial culture with bacteriophages. Examples of bacteriophages include, but not limited to, φadh, φLC3, mv4, M13, T4, φ29, Cp-1, Cp-7, and Cp-9. Before ingestion of the bacterial culture, the bacterial cells may be infected with sufficient amount of bacteriophages. Because there is a lag time between the infection and lysis, there should be enough time for the bacterial cells to travel down to the intestines before lysis occurs. Alternatively, the bacteriophage may be introduced hours or days after the first ingestion. In this latter embodiment, a first ingestion of the bacterial culture is allowed to colonize the intestines and multiply in number. A second culture of bacteria infected with the bacteriophages is then administered hours or days after the first culture. When the bacteriophages lyse the cells in the intestines, bacteriophage particles may further infect the bacteria cells in the intestinal mucosa and increase more plasmid DNA in the intestine.

In another embodiment, the LAB strain may be genetically engineered to express an autolysing gene under the control of an inducible promoter. The autolysing gene may then be triggered at the appropriate time and place in the gastrointestinal tract through the use of inducible promoters and use to lyse the bacterial cells. Examples of autolysing gene include, but not limited to, AcmA (Buist G., et al., (1997) “Autolysis of Lactococcus lactis caused by induced overproduction of its major autolysin, AcmA,” Appl. Environ. Microbiol, 63:2722-2728); holin and lysin (Henrich, B., et al., (1995) “Primary Structure and Functional Analysis of Lysis Genes of Lactobacillus gasseri Bacteriophage φadh,” J. Bacteriology, Vol.177, No. 3, 723-732), which are hereby incorporated by reference.

Preferably, the lysis occurs in the intestine of the body such that the intestinal cells may then take up the plasmid. Examples of inducible promoters for used with the autolysing gene include, but not limited to, a pH inducible promoter as described in U.S. Pat. No. 6,242,194 issued to Kullen, et al., which is hereby incorporated by reference as if fully set forth herein; a lactose inducible promoter such as that used in E. coli plasmds (e.g. pBluescript from Stratagene) or the endogenous lactose promoter in lactobacillus; promoters induced during anaerobic growth such as the promoter for alcohol dehydrogenase (adhE), as described in Aristarkhov, A. et al., “Translation of the adhE Transcript to Produce Ethanol Dehydrogenase Requires Rnase III Cleavage in Escherichia coli,” J. Bacteriology, Vol. 178, No.14, 4327-4332. In the case of lactose, inducing the autolysing gene may involve taking lactose such as present in milk or yogurt after ingestion of the bacterial culture. Lactose may be introduced hours or even a few days after ingestion to allow the recombinant LAB to further multiply in number in the intestines. In the case of adhE promoter, the promoter is induced in the intestines because of the anaerobic conditions in the intestines.

The autolysing gene and the inducible promoter, together as an expression cassette, may be part of a plasmid that is transformed in the bacterial strain. On the other hand, the autolysing gene expression cassette may be preferably integrated into the chromosomal DNA of the LAB strain to be used. Integration into the chromosome may be achieved by flanking DNA sequences on both sides of the autolysing gene expression cassette wherein the flanking DNA sequences are homologous to a targeted sequence in the chromosome. The entire construct comprising the flanking DNA sequences and the autolysing gene may then be used to transform the microflora. Through homologous recombination, the autolysing gene may then be integrated into the genome of the LAB. The described method of modifying bacterial strains through homologous recombination has been used extensively in E. coli cells and may be applied likewise to LAB.

The nucleotide sequences encoding the antigen or therapeutic element and the surface binding promoter regions may be prepared in a variety of ways. These sequences can be obtained from any natural source or may be prepared synthetically using well-known DNA synthesis techniques. The sequences can then be incorporated into a plasmid, which is then used to transform the chosen bacterial host. Recently, advances in molecular biology with respect to recombinant production of proteins has made it possible to express foreign proteins at the outer surface of microorganisms by the technology called cell surface display. Sequences for surface binding promoter regions will be fused to the sequence of the antigen, such that the modified lactobacillus organism will present the antigen on its surface. Examples of such surface binding promoter regions are those used in the construct described in PCT/NL96/00135 and those described in Dieye, Y., et al., “Design of a protein-targeting system for lactic acid bacteria” Journal of Bacteriology 183(14); 4157-66 (2001).

One of the first surface-expression systems was developed in the mid 1980s by George P. Smith. He was able to express peptides or small proteins fused with pill of the filamentous phage (see: Smith, G. P., Science, 228:1315-1317, 1985). Following that time, various systems of heterologous protein expression and secretion in microorganisms have been studied to develop new and better cell surface display and secretion systems by which proteins of interest can be expressed on the surface of the microorganisms or secreted. Using endogenous surface proteins, as a surface anchoring motifs, the current inventor has been studying the use of bacteria and yeast for the stable expression of proteins or peptides on the surface of a cell.

Bacteria, especially gram-negative bacteria such as E. coli, possess unique and complex cell envelope structures that may consist of an inner cellular membrane, periplasm, and outer cellular membrane. Hence, to efficiently transport foreign proteins to the cell surface a surface anchoring motif is needed. Therefore, in order to express a foreign peptide or protein, an appropriate bacterial surface protein has to be fused to the foreign protein of interest, at the genetic level, and the fusion protein expressed has to be transported through the inner cellular membrane and outer membrane to the surface of the bacteria where it then becomes anchored.

Given these factors, a surface anchoring motif needs to have several key characteristics. First of all, the surface protein to be used as an anchoring motif needs to have a sufficient secretion signal sequence motif to allow the transport of the foreign protein through the inner membrane of the cell. Secondly, a targeting signal for anchoring the foreign protein to the surface of the cell is also needed. Additionally, the overall fusion motif needs to have the capacity to not only accommodate foreign proteins or peptides of various sizes but to also express them in large amounts.

There are basically three groups of cell surface display systems that have been developed: C-terminal fusion, N-terminal fusion, and sandwich fusion. First of all, if a native surface protein has a discrete localization signal within its N-terminal portion, a C-terminal fusion motif may be used to fuse a foreign peptide to the C-terminal of that functional portion. For example, the Lpp-OmpA motif developed in E. coli uses a C-terminal fusion system (see: Georgiou, G., et al., Protein Eng., 9:239-247, 1996). Secondly, a N-terminal fusion motif has been developed which contains a C-terminal sorting signals to target foreign proteins to the cell wall. Examples of bacteria for which an N-terminal fusion motif has been developed include the Staphylococcus aureus protein A (see: Gunneriusson, E., et al., J. Bacteriol., 178:1341-1346, 1996), Staphylococcus aureus fibronectin binding protein B (see: Strauss, A., et al., Mol. Microbiol., 21:491-500, 1996), and Streptococcus pyogenes fibrillar M protein (see: Pozzi, G., et al., Infect. Immun., 60:1902-1907, 1992.). However, if the surface proteins do not have such anchoring regions the whole structure will be required for assembly. For this reason, a sandwich-fusion system has been developed, in which a foreign protein of interest is inserted into the surface protein motif. Several examples employing this system include E. coli PhoE (see: Agterberg, M., et al., Gene, 88:37-45, 1990), FimH (see: Pallesen, L., et al., Microbiology, 141:2839-2848,1995), and PapA (see: Steidler, L., et al., J. Bacteriol., 175:7639-7643, 1993). Using these mechanisms, a person of ordinary skill in the art will be able to modify a given expression system for a given bacterium such as Streptococcus and Lactococcuss so as to effect the purposes of the present invention, namely expression, secretion and/or cell surface display of various antigenic and/or therapeutic elements.

For secretion of the translated protein into the extracellular environment, appropriate secretion signals may be incorporated into the desired polypeptide. These signals may be endogenous to the polypeptide or they may be heterologous signals. Hence, secretion signals may be used to facilitate delivery of the resulting protein. The coding sequence for the secretion peptide is operably linked to the 5′ end of the coding sequence for the protein, and this hybrid nucleic acid molecule is inserted into a chosen plasmid adapted to express the protein in the host cell of choice. Plasmids specifically designed to express and secrete foreign proteins are available from commercial sources. For example, if expression and secretion is desired using an E. coli expression system, commonly used plasmids include pTrcPPA (Pharmacia); pPROK-C and pKK233-2 (Clontech); and pNH8a, pNH16a, pcDNAII and pAX (Stratagene), among others. Other secretion signal systems are those such as the M6 preprotein from Streptococcus pyrogens described in Dieye, Y., et al., “Design of a protein-targeting system for lactic acid bacteria” Journal of Bacteriology 183(14); 4157-66 (2001) and those set forth, such as SP13, SP10, SP307 and SP310 recognized by signal peptidase I or II, in Ravn, P., et al., “The Development Of TnINuc And Its Use For The Isolation Of Novel Secretion Signals In Lactococcus Lactis” Gene 242: 347-356 (2000).

Hence, in one embodiment the invention embodies methods for producing heterologous proteins in a host organism whereby the protein is processed through the secretory pathway of the host. Secretion is achieved by transforming a host organism, i.e., E. coli, with a plasmid containing a DNA construct comprising a transcriptional promoter operably linked to DNA sequences encoding a secretion signal peptide, for instance the portion of the BAR1 C-terminal domain or the Staphylococcus aureus protein A that is capable of directing the export of heterologous proteins or polypeptides.

Examples of other various secretion systems described for use in E. coli include U.S. Pat. No. 4,336,336 (filed Jan. 12, 1979); European Pat. Application Publication Numbers 184,169 (published Jun. 11, 1986), 177,343 (published Apr. 9, 1986) and 121,352 (published Oct. 10, 1984); Oka, T. et al. (1985); Gray, G. L. et al. (1985); Ghrayeb, J. et al. (1984) and Silhavy, T. et al. (1983). For the most part, these systems make use of the finding that a short (15-30) amino acid sequence present at the amino NH₂-terminus of certain bacterial proteins, which proteins are normally exported by cells to noncytoplasmic locations, are useful in similarly exporting heterologous proteins to noncytoplasmic locations. These short amino acid sequences are commonly referred to as “signal sequences” as they signal the transport of proteins from the cytoplasm to noncytoplasmic locations. In Gram-negative bacteria, such noncytoplasmic locations include the inner membrane, periplasmic space, cell wall and outer membrane. At some point just prior to or during transport of proteins out of the cytoplasm, the signal sequence is typically removed by peptide cleavage thereby leaving a mature protein at the desired noncytoplasmic location. Site-specific removal of the signal sequence, also referred to herein as accurate processing of the signal sequence, is a preferred event if the correct protein is to be delivered to the desired noncytoplasmic location.

Hence, in one embodiment the present invention relates to a Streptococcus thermophilus or Lactococcus lactis organism that is modified by fusion with an E. coli bacteria that contains a plasmid encoding a heterologous nucleic acid that is operably linked to a promoter capable of driving expression of the genetic element in the modified host bacteria. According to one particular embodiment, the heterologous nucleic acid is polynucleotide sequence coding for an antigen that is either capable of being secreted or displayed on the cell surface of the bacteria. In either case, the plasmid encoding the heterologous nucleic acid will also contain, the appropriate secretion or anchor sequence information required for either secretion or cell surface delivery and expression. According to this embodiment, the protein or peptide fragment produced within the fusant comprises an antigen capable of eliciting an immune response when it comes into contact with an immune related cell of the body.

In the case wherein the protein is secreted, the related immune cell is expected to be a secreted IgA antibody, however, it is also likely that the secreted antigenic fragment may be endocytosed by the M cells of the Peyer's patches, in which case the antigenic protein or fragment may come into contact with the various components of the M cell pocket, including CTLs, B cells, macrophages and dendritic cells, thereby inducing a mucosal immune response. In the case where the protein or antigenic fragment is anchored and displayed on the cell surface of the fusant, the antigenic fragment may come into direct contact with the cell surface membrane of the M cells thereby directly interacting with the various components of the M cell directly to illicit a mucosal immune response.

According to another particular embodiment, the heterologous nucleic acid is polynucleotide sequence coding for a therapeutic protein or peptide fragment that is either capable of being secreted or displayed on the cell surface of the bacteria. In either case, the plasmid encoding the heterologous nucleic acid will also contain the appropriate secretion or anchor sequence information required for either secretion or cell surface delivery and expression. According to this embodiment, the protein or peptide fragment produced within the fusant comprises a therapeutic such that when it is expressed it produces a protein or fragment thereof necessary for modifying and or correcting a diseased state. Particularly, the heterologous nucleic acid encodes a protein capable of being secreted into the lumen of the respiratory tract, such as insulin, whereby when the protein is secreted it is capable of being absorbed and modifying a diseased state, such as diabetes.

As mentioned briefly above, fusants comprising bacteria-bacteria can also be used in accordance with the teachings of the present invention. Several different bacteria with suitable expression systems can be fused with a non-pathogenic Streptococcus or Lactococcus bacteria to generate the desired modified LAB organism. In a preferred embodiment of the invention, Streptococcus or Lactococcus bacteria are fused with Escherichia coli (E. coli). Several different strains of E. coh that are commonly used for molecular cloning are HB101, C600, DH1, DH10B, DH5 α5 and β10. The strains mentioned are preferred because well-defined and commercially available expression systems for the production and expression of heterologous nucleic acids are already available for them.

In one embodiment of the invention, bacteria of one species are fused with bacteria of a different species. Two particular species of bacteria that have reported expression systems are Lactococcus lactis and Bacillus subtilis. Cocconcelli, PS, et al. “Single-stranded DNA plasmid, vector construction and cloning of Bacillus stearothermophilus alpha-amylase in Lactobacillus” Research in Microbiology 142(6): 643-52 (1991) and Kleerebezem, M., et al. “Controlled gene expression systems for lactic acid bacteria: transferable nisin-inducible expression cassettes for Lactococcus, Leuconostoc, and Lactobacillus spp.” Applied and Environmental Microbiology 63(11): 4581-84 (1997).

In one embodiment, the expression system of the present invention will contain a DNA construct comprising at least a nucleotide sequence encoding a desired antigen or therapeutic gene operably linked to a promoter that can direct expression of the heterologous sequence in a bacterial host. The polynucleotide encoding the antigenic or therapeutic fragment may include the coding sequence for the mature polypeptide or a fragment thereof, by itself or the coding sequence for the mature polypeptide or fragment in reading frame with other coding sequences, such as those encoding origin(s) of replication, an anchor, leader or secretory sequence, a pre-, or pro- or prepro-protein sequence, or other fusion peptide portions. For example, a marker sequence which facilitates selection of the fused polypeptide can be encoded. The polynucleotide may also contain non-coding 5′ and 3′ sequences, such as transcribed, non-translated sequences, splicing and polyadenylation signals, ribosome binding sites and sequences that stabilize mRNA.

In one embodiment, a LAB, such as the species thermophilus or lactis, is fused with E. coli in such a way as to allow the thermophilus or lactis bacteria to express an antigenic or therapeutic protein or polypeptide encoded by the E. coli associated DNA. Preferably, the antigenic polypeptide is capable of being expressed on the cell surface of the LAB-E. coli fusant, while the therapeutic protein is capable of being secreted. Hence, it is often advantageous to include an additional polynucleotide sequences coding for the amino-acid sequences which contain anchor, secretory or leader sequences, or additional sequences for stability during in vivo production. The protein of polypeptide fragments produced are then capable of either being expressed on the LAB-E. coli fusant's cell surface, or secreted, and thereby eliciting either an immune or therapeutic response.

Representative polypeptide fragments include, for example, those coding for antigenic epitopes capable of being recognized by the various immune initiating cells of the body, specifically, M cells, IgA and IgG cells, i.e., they are antigenic or immunogenic in an animal, especially in a human. Variants of the defined sequence and fragments also form part of the present invention. Preferred variants are those that vary from the referents by conservative amino acid substitutions. Other preferred fragments include biologically active, therapeutic fragments that mediate activity, including those with a similar activity or an improved activity, or with a decreased undesirable activity. Preferably, these polypeptide fragments retain the biological activity of the antigen or therapeutic, including antigenic activity.

Hence, in one particular embodiment the present invention relates to E. coli derived vectors that contain an antigenic or therapeutic polynucleotide or polynucleotides, host Streptococcus thermophilus or Lactococcus lactis cells that are genetically engineered by fusion with E. coli cell vectors, and to the production and expression of the encoded antigenic or therapeutic polypeptides by the host LAB cell-E. coli fusants. Suitable E. coli cells with appropriate expression systems can be purchased from various commercial sources, or genetically engineered, and made to incorporate expression systems or portions thereof for antigenic or therapeutic polynucleotides of the present invention.

Introduction of the polynucleotides into E. coli cells can be effected by methods described in many standard laboratory manuals, such as Davis et al., Basic Methods In Molecular Biology (1986) and Sambrook et al., Molecular Cloning: A Laboratory Manual, 2nd Ed., Cold Spring Harbor Laboratory Press, Cold Spring Harbor, N.Y. (1989) such as calcium phosphate transfection, DEAF-dextran mediated transfection, microinjection, cationic lipid-mediated transfection, electroporation, transduction, scrape loading, ballistic introduction or infection. Representative examples of appropriate LAB hosts for fusion with the E. coli cells and the in vivo production of antigenic and therapeutic proteins and/or polypeptides include Streptococcus thermophilus or Lactococcus lactis as well as Lactobacillus bacterial cells, such as: acidophilus, brevis, casei, delbrueckii, fermentum, or plantarum.

More particularly, the present invention includes recombinant E. coli vectors into which an antigenic and/or therapeutic construct comprising a DNA, cDNA or RNA sequence has been inserted, in a forward or reverse orientation. In a preferred aspect of this embodiment, the construct further comprises regulatory sequences, including, for example, a promoter, operably linked to the genetic sequence. Large numbers of suitable plasmids and promoters are known to those of skill in the art, and/or described below, and are also commercially available.

Hence, in one embodiment of the invention, the DNA construct will be a plasmid encoding at least an appropriate origin of replication for the desired bacterial host, a selectable marker gene and/or a reporter gene, a promoter operably linked to a heterologous nucleotide sequence encoding the antigen or therapeutic element fused to surface binding promoter or anchor region. The construct may also contain other suitable elements, such as transcription initiation sequences, secretion signal sequences and transcription termination sequences. Plasmids will be chosen or created based on their ability to replicate- in the host bacteria. Where the expression system is derived from E. coli, plasmid vectors into which the promoter and nucleotide sequence could be cloned include, for example pUC18, pUC19, pBR322, and pBluescript. For LAB appropriate plasmids include, for example, pFXL03, pWV01, pGKV210 and. pVA838. Some plasmids from Lactobacillus and Lactococcus can be obtained from DSMZ, Braunschweig, Germany. Others have been described in the literature. For example, plasmid vectors suitable for Lactobacillus casei are described in many references, including Maassen, C., et al., “Instruments for oral disease-intervention strategies: recombinant Lacrobacillus casei expressing tetanus toxin fragment C for vaccination or myelin proteins for oral tolerance induction in multiple sclerosis” Vaccine 17(17): 2117-28 (1999). In addition, plasmid vectors suitable for Lactobacillus plantanun and Lactococcus lactis are described in Geoffrey, M., et al., “Use of green fluorescent protein to tag lactic acid bacterium strains under development as live vaccine vectors” Applied and Environmental Microbiology 66(1): 383 (2000). Plasmid vectors for Lactococcus lactis, Lactobacillus fermentum, and Lactobacillus sake are described in Piard, J., et al., “Cell wall anchoring of the Streptococcus pyogenes M6 protein in various lactic acid bacteria” Journal of Bacteriology 179(9): 3068-72 (1997). Some plasmid vectors are suitable for a wide range of Lactobacillus species, such as pPSC20 and pPSC22, described in Cocconcelli, P., et al., “Single-stranded DNA plasmid, vector construction and cloning of Bacillus stearothermophilus alpha-amylase in Lactobacillus” Res Microbiol 142(6): 643-52 (1991). Shuttle vectors, which are plasmids that are capable of expression in both of the parent bacteria used to create the fusant, could also be used. In this instance, appropriate shuttle vectors would contain origins of replication from both fusant species. Appropriate shuttle vectors for LAB include pFXL03, pWV01, pGKV210, pVA838, pNZ123 etc. Furthermore, Lactobacillus/E. coli shuttle vectors are described in Maassen, C., et al., Vaccine, ibid. and Bringel, et al. “Characterization, cloning, curing, and distribution in lactic acid bacteria of pLP1, a plasmid from Lactobacillus plantarum CCM 1904 and its use in shuttle vector construction” Plasmid 22(3): 193-202 (1989).

The plasmid could contain either selectable marker genes or reporter genes used to facilitate determining which bacteria contain the desired plasmid DNA. Possible selectable marker genes are antibiotic resistance markers, such as kan^{r}, tetr and ampr. The gene for Beta galactosidase and the gene encoding green fluorescent protein (GFP) are examples of reporter genes. Alternatively, if the plasmid does not include a selectable marker or reporter gene the plasmid DNA could be detected in a variety of ways, such as a dot blot using the plasmid DNA as a probe.

The choice of promoter will depend on the host bacteria and the antigen to be expressed. Promoters that could be used with E. coli expression systems include lambda PR, PL and trp, as well as T3, T7, gpt, SP6 and the lacZ promoter or Lac operon. Promoters for various Lactobacillus and Lactococcus bacteria have been described in the literature. For example, one promoter that is well suited for both Lactobacillus plantarun and Lactococcus lactis and that has also been shown to be useful for expression in other LAB is the nisin inducible nisA promoter from Lactococcus lactis. (deRuyter, P., et al., “Controlled gene expression systems for Lactococcus lactis with the food-grade inducer nisin” Appl. Environ. Microbiol. 62: 3662-67 (1996)) Kleerebezem, M., “Controlled gene expression systems for lactic acid bacteria: transferable nisin-inducible expression cassettes for Lactococcus, Leuconostoc, and Lactobacillus spp.” Applied and Environemntal Microbiology 63(11): 4581-84(1997)) The L. plantarum 1dhL promoter has also been used successfully in L. plantarum. Promoters for L. casei expression systems include the constitutive lactate dehydrogenase promoter from L. casei and the regulatable amylase promoter from L. amylovorus (Maassen, C., et al., Vaccine 17(17): 2117-28 (1999)). The lactococcal promoter P₅₉ has been used in expression vectors of various Lactococcus lactis and Lactobacillus bacteria (Piard, J., et al., “Cell wall anchoring of the Streptococcus pyogenes M6 protein in various lactic acid bacteria” Journal of Bacteriology 179(9): 3068-72 (1997)). In addition, the plasmids could contain multiple promoter sequences all operably linked to the sequence encoding the antigen. Each of the promoters in such a vector would be compatible with at least one of the parent bacteria used to make the fusant, furthermore, as mentioned, the plasmids may contain multiple origins of replication, such as that from each parent species.

If an antigenic polypeptide is to be expressed, generally, it is preferred that the polypeptide be produced and displayed at the surface of the cell. Recently, advances in molecular biology with respect to recombinant production of proteins has made it possible to express foreign proteins at the outer surface of microorganisms by the technology called cell surface display. Sequences for surface binding promoter regions will be fused to the sequence of the antigen, such that the modified Lactobacillus organism will present the antigen on its surface. Examples of such surface binding promoter regions are those used in the construct described in PCT/NL96/00135 and those described in Dieye, Y., et al., “Design of a protein-targeting system for lactic acid bacteria” Journal of Bacteriology 183(14); 4157-66 (2001).

Saccharomyces cerevisae is yeast that is considered to be compatible for use in human expression systems because of such similarities such as cell cycle, chromosomal structure and RNA-splicing. The current inventor has chosen to develop an expression system using this yeast because of its ability to perform post-translational modifications such as acetylation, phosphorylation and glycosylation of proteins produced in a fairly similar manner as to that in animal cells. Therefore, use of S. cerevisae as a host for expression of a foreign proteins in an animal, specifically a human, is expected to provide a gene product closer to its natural form, like that produced by animal cells. Because S. cerevisae has a lot in common with both other yeast and bacteria, with respect to their culture methods, knowledge about the use of other microflora, and the microbiological methods and DNA recombination techniques used to manipulate them, can be easily applied for production of a foreign proteins within the present system using S. cerevisae.

Various proteins, especially those used as pharmaceuticals have been produced in yeasts, including the genus Saccharomyces, whose safety has been widely recognized (Marten & Seo, Chap. 7, Expression Systems and Processes for rDNA Products, ed. by Hatch et al., ACS Symp. Ser., 477 (1991)). The expression and secretion vectors used to produce and secrete desired proteins from yeast comprise a plasmid with a transcription promoting sequence (promoter), a DNA encoding a secretion signal peptide, a structural gene encoding a desired protein, and a transcription terminator, as well as a reporter gene, if necessary. In the situation where it is desirable for the protein or peptide fragment to be displayed on the cell surface of the yeast fusant, the protein or peptide encoding sequences are fused with a secretion signal and an appropriate anchor sequence on the 5′ and 3′-ends, respectively.

As the transcription promoting sequence (promoter) for such vectors, there have been used GAPDH, MF.alpha.-1 promoter or PGK (Loison et al., Korean Patent Laid-open Publication Nos. 88-7727 and 88-700234; Bio/Technol., 6, 72 (1988)), GAPDH, mating factor-.alpha. (MF.alpha.-1), PH05 (Meyhack et al., Korean Patent Laid-open Publication Nos. 86-381 and 87-6185; Genetics and Molecular Biology of Industrial Microorganisms, ed. by Hershberger et al., published by American Society of Microbiology, pp. 311-312 (1989)), and GAL promoter series (Johnston, Microbiol. Rev., 51, 458-476(1987)) such as GALL which are induced by galactose in the culture medium.

With respect to secretion signals, secretion signal peptides currently used for the secretion of heterologous proteins from yeast include an invertase signal peptide (U.S. Pat. No. 5,010,003), an acid phosphatase signal peptide (U.S. Pat. No. 5,013,652), a prepro leader peptide (ppL) of mating factor-.alpha. (U.S. Pat. No. 4,588,684). Among these various secretion signal peptides, the ppL is most widely used. With respect to cell wall anchor sequences, various sequences that could be fused (in conjunction with yeast secretion signals) to the antigen to effect cell surface display on the cell wall include the carboxy terminal parts of Gas1p (sequence encoding last 252 amino acids) or Yap3p (sequence encoding last 35 amino acids). For details, see de Sampaio, G., et al., “A constitutive role for GPI anchors in Saccharomyces cerevisiae: cell wall targeting” Molecular Microbiology 34(2): 247-56 (1999). See also Van Der Vaart et al. “Comparison of cell wall proteins of Saccharomyces cerevisiae as anchors for cell surface expression of heterologous proteins” Applied and Environmental Microbiology 63(2):615-620 (1997).

Hence, in one particular embodiment, the present invention relates to a Saccharomyces organism that is modified by fusion with an E. coli bacteria that contains a plasmid encoding a heterologous genetic element that is operably linked to a promoter capable of driving expression of the genetic element in the modified host yeast strain. Various promoters utilized by RNA polymerase II may be used, they may be inducible, such as GAL, GAL10, PH05 and the like, or they may be constitutive, such as ADH1, TPI, PGK and the like. Furthermore, the various vectors that may be used include, but not here limited to: PBM150, PYEP51, PLGSD5, YEP62, PAAH5 and the like. Furthermore, the plasmid coding for the heterologous genetic element may also contain a selectable marker such as URA3, LEU2, HIS3, TRPI and the like.

According to one particular embodiment, the heterologous genetic element is polynucleotide sequence coding for an antigen that is either capable of being secreted or displayed on the cell surface of the bacteria. In either case, the plasmid encoding the heterologous genetic element will also contain the appropriate secretion or anchor sequence information required for either secretion or cell surface delivery and expression. According to this embodiment, the protein or peptide fragment produced within the fusant comprises an antigen capable of eliciting an immune response when it comes into contact with an immune related cell of the body.

According to another particular embodiment, the heterologous genetic element is polynucleotide sequence coding for a therapeutic protein or peptide fragment that is either capable of being secreted or displayed on the cell surface of the bacteria. In either case, the plasmid encoding the heterologous genetic element will also contain the appropriate secretion or anchor sequence information required for either secretion or cell surface delivery and expression. According to this embodiment, the protein or peptide fragment produced within the fusant comprises a therapeutic such that when it is expressed it produces a protein or fragment thereof necessary for modifying and or correcting a diseased state. Particularly, the heterologous genetic element encodes a protein capable of being secreted into the lumen of the intestinal mucosa, such as insulin, whereby when the protein is secreted it is capable of being absorbed and modifying a diseased state, such as diabetes.

In one embodiment of the invention, the recombinant LAB, fusants and yeasts may be targeted to M-cells, such as those associated with Peyer's patches in the intestinal mucosa. Within the mucosal epithelial lining, bits of lymphoid tissue make up the organized mucosal lymphoid follicle-associated epithelium (FAE) tissue. Though the epithelium that lines the intestinal mucosa is impermeable to macromolecules and microorganisms, in mucosal inductive sites, such as the Peyer's patches in the intestinal tract, the lymphoid FAE contains microfold, or M-cells, that allow the transportation of antigens and microorganisms, for antigen sampling. M-cells, in simple epithelia only occur over organized lymphoid follicles. Hence, at FAE sites, rich in M-cells, there is a highly developed collaboration of the specialized epithelia with antigen-presenting and lymphoid cells. Through active trans-epithelial vesicular transport, M-cells transport macromolecules, particles, and microorganisms from the lumen, across their cytoplasm and directly into the intraepithelial mucosal lymphoid follicles and to organized mucosal lymphoid tissues that are designed to process antigens and initiate a mucosal immune response that results in secretory immunity—the process by which mucosal surfaces of the intestinal mucosa and lung are bathed with protective antibodies. The lymphoid tissues are, therefore, also the gateway into the circulation of the antigens, cells, antibodies, and any other proteins in the lymphatic system and ultimately the blood.

Hence, M-cells provide local, functional openings in the epithelial barrier through which vesicular transport occurs. Restriction of M-cells to the sites directly over lymphoid follicles (FAE) serves to reduce the inherent risk of transporting foreign material and microbes across the epithelial barrier by assuring immediate exposure to phagocytes and antigen-presenting cells. The apical surfaces of M-cells, facing the lumen, are distinguished from neighboring cells by the absence of a typical brush border and the presence of variable microvilli or microfolds with large intermicrovillar endocytic domains. A basal invagination in M-cells creates a unique feature of the M-cell, which is an intraepithelial “pocket” or space that both shortens the distance that transcytotic vesicles must travel from the apical to the basolateral surface and provides a docking site for lymphocytes, such as B and CD4 T cells, macrophages and dendritic cells to gather. M-cells also have basal processes that extend more into the underlying lymphoid tissue where they make direct contact with lymphoid and/or antigen-presenting cells, which likely plays a role in the presentation of antigens after M-cell transport.

M-cells engage in several different modes of transcytosis for the transport of foreign material into endosomal tubules, vesicles and large multivesicular bodies in the M-cell apical cytoplasm and to their subsequent release by exocytosis into the pocket. Adherent viruses and macromolecules are taken up by adsorptive endocytosis via clathrin-coated pits and vesicles. Non-adherent materials are taken up by fluid-phase endocytosis in either coated or uncoated vesicles. Large adherent particles and bacteria trigger phagocytosis, involving the extension of cellular processes and the reorganization of the submembrane actin network.

The ability of M-cells to conduct transport of intact macromolecules from one side of the barrier to the other involves the directed movement of membrane vesicles. Although the molecular mechanisms of this transport have not been determined in M-cells, it is safe to assume that the membrane traffic conducted by M-cells depends on the polarized organization and signaling networks typical of polarized epithelial cells. M-cells are unique among epithelial cells in that trans-epithelial vesicular transport is the major pathway for endocytosed materials. Studies have shown that endocytic vesicles formed at the apical surface of M-cells first deliver their cargo to endosomes in the apical cytoplasm and that these acidify their content and contain proteases.

One of the primary components in the M-cell pocket is B cells. The B cells in the pocket express IgM but not IgG or IgA, suggesting that B memory cells and/or initial B cell differentiation may occur here. The presence of memory phenotypes suggests that cells in the pocket have positioned themselves for re-exposure to incoming antigens. It has been suggested that B lymphoblast traffic into the M-cell pocket may allow for repeated antigen exposure and extension and diversification of the immune response. However, immediately under the FAE, there is an abundance of other B lymphoblasts, helper T cells, and antigen-presenting cells that are sufficient for initiating an immune response.

Lumenal antigens transcytosed by M-cells are immediately delivered to these antigen-processing and -presenting cells that then migrate to antigen-specific lymphocytes, in the underlying lymphoid follicles located in the mucosa-associated lymphoid tissue (MALT), which further induces their proliferation. Thus, passage of antigens and microorganisms through M-cells is an essential step for the development of mucosal immune responses. This process results in the development of IgA-producing B cells, some of which move into the vasculature and then back to the mucosal surfaces, efficiently seeding specific mucosal immunity.

The mucosal immune system consists of the specialized local inductive sites (the Organized Mucosa Associated Lymphoid Tissue or O-MALT) and widespread effector sites (the Diffuse Mucosa Associated Lymphoid Tissue or D-MALT), both of which are separated from mucosal surface antigens by epithelial barriers. The first step in the induction of a mucosal immune response is the transport of antigens across the epithelial barrier. Following antigen processing and presentation in inductive sites, IgA-committed, antigen-specific B lymphoblasts proliferate locally and then migrate via the bloodstream to local and distant mucosal and secretory tissues. There they differentiate primarily into polymeric IgA-producing plasma cells, which are important components of D-MALT, and are transported across epithelial cells into glandular and mucosal secretions via receptor-mediated transcytosis.

Hence, mucosal immunity forms a first line of defense against mucosally transmitted pathogens such as influenza and is important for long-term protection. Mucosal defense against pathogens consists of both innate barriers, such as mucous, epithelium, and innate immune mechanisms, and adaptive host immunity, which at mucosal surfaces consists predominantly of CD4+ T cells, secretory immunoglobulin A (S-IgA), and antigen-specific cytotoxic T-lymphocytes (CTLs). Under healthy circumstances, transport by M-cells and the resulting secretion of antimicrobial sIgA antibodies limit the intensity or duration of mucosal disease and prevent reinfection.

By targeting the modified microflora to the M-cells of the intestinal lining, there is an increased chance that the expression of an antigenic protein from the eukaryotic plasmid are presented to the immune cells in the M-cell pockets. Similarly, expression of any therapeutic proteins such as insulin, growth hormone, interferon, etc., may be secreted through the M-cell pocket into the lymphoid tissue and into the blood circulation. Although M-cells are preferred for targeting, the plasmid DNA may also be delivered to other cells in the intestines such as K-cells and other rapid dividing epithelial cells. In fact, it has been shown that expression of Factor VIII and IX, for example, in rapidly dividing epithelial cells preferentially secretes Factor VIII and IX in the basolateral direction (i.e., away from the lumen and toward underlying capillaries and lymphatics). Consequently, it is expected that expression of secreted therapeutic proteins in M-cells, K-cells or other intestinal cells will lead to the proteins actually being found in the blood stream and in circulation. See Lozier, JN, “Gut epithelial cells as targets for gene therapy of hemophilia,” Hum Gene Ther (1997) Aug 10;8(12):1481-90. To further increase the targeting of the proteins for basolateral secretion, targeting signal sequence may be used such as the basolateral-specific 11 amino acid signal sequence from the glycoprotein G of vesicular stomatitis virus. Thomas and Roth, “The Basolateral Targeting Signal in the Cytoplasmic Domain of Glycoprotein G from Vesicular Stomatitis Virus Resembles a Variety of Intracellular Targeting Motifs Related by Primary Sequence but Having Diverse Targeting Activities,” J. Biol. Chem., Vol. 269, No. 22,15732-15739 (1994).

On the other hand, certain proteins may be preferred to be secreted in the lumen such as nisins, which are naturally occurring bacteriocins expressed by lactobacillus. In such as case, an apical surface specific signal sequence may be used. For example, glycosylphosphatidylinositol (GPI) attachment sequence from Thy-1 may be fused with the protein of interest to target a particular protein to the apical layer in the intestinal mucosal cells. The GPI attachment sequence, for example, has been shown to result in 2.5 times more apical delivery of expressed proteins than basolateral delivery in polarized intestinal Caca-2 cells in culture. See Soole, K., et al., “Epithelial Sorting of a glycosylphosphatidylinositol-anchored bacterial protein expressed in polarized renal MDCK and intestinal Caco-2 cells,” Journal of Cell Science, 108, 369-377 (1995), which is hereby incorporated by reference.

M-cell targeting may be accomplished in a variety of ways, including using compounds that bind to M-cell surface compounds. Such compounds include polypeptides, such as M-cell receptors or surface antigens, carbohydrates, and glycoconjugates. M-cell targeting may involve compounds that specifically bind to M-cells as well as compounds that specifically bind to cells of tissue with which M-cells are associated, such as the epithelial cells of the intestinal mucosa.

One example of a compound that binds to M-cells are adhesins from bacteria and viruses that target M-cells, such as the Yersinia species and Salmonella typhi, respectively. (Clark, M. A., et al., “M-cell surface β1 integrin expression and invasin-mediated targeting of Yersinia pseudotuberculosis to mouse Peyer's patch M-cells” Infect Immun. 66:1237-43 (1998); Baumler, A. et al., “The Ipf fimbrial operon mediates adhesion of Samonella typhirium to murine Peyer's patches” Proc. Natl. Acad. Sci. USA 93: 279-83 (1996). Such bacterial and viral adhesins are proteins that mediate M-cell binding. The protein sigma 1 of the reovirus has also been used to target M-cells. Wu, Y., et al., “M-cell-targeted DNA vaccination” Proc. Natl. Acad. Sci. USA 98(16): 9318-23 (2001).

Another compound that binds specifically to M-cells is lectin. M-cell targeting of lectin bearing-liposomes to M-cells using various types of lectin is described in U.S. Pat. No. 6,060,082, which is incorporated herein by reference.

Antibodies that bind specifically to M-cell surface proteins such as receptors or surface antigens may also be used for M-cell targeting. Antibodies to such surface proteins can be generated in a variety of ways that are well known in the art, using the entire protein of interest (either the precursor or the processed protein) or a portion thereof. Furthermore, the sigma protein from reovirus, which targets M-cells may also be used. See Wu, Y., et al., “M-cell Targeted DNA Vaccination,” PNAS Vol. 98, No. 16, 9318-9323 (2001).

The M-cell targeting compounds described above can be incorporated into the cell wall of the modified microflora. This can be accomplished by adding the M-cell targeting compound to protoplasts, for example, during the process of regenerating cell walls. Preferably, the M-cell targeting compound is derivatized to lipids designed to act as membrane anchors. Such functionalized lipids can be purchased from Avanti Polar Lipids, Inc. (Alabaster, Ala.).

Alternatively, a plasmid could encode an M-cell targeting polypeptide. In one embodiment the plasmid containing the sequence for the antigen or the therapeutic protein to be expressed also contains the sequence for the M-cell targeting polypeptide. Alternatively, the M-cell targeting polypeptide sequence could be encoded by a piece of DNA that has been homologously recombined and integrated into the genome of the microflora, similar to the method described above for integrating the autolysing gene into the chromosome.

The following examples illustrate methods and compositions to be used according to the present invention. These examples are provided for illustrative purposes only and shown not be construed as limiting the scope of the invention. Although the examples may be described with reference to L. acidophilus strains—which are most preferred—strains of other Lactobacillus species, LAB and E. coli may also be suited for use according to the invention.

Methods in microbiology, molecular biology, and cell culture mentioned but not explicitly described in this disclosure have already been amply reported in the scientific literature. These methods are well within the ability of one skilled in the art.

### EXAMPLE I

### Formation of Lactobacillus acidophilus Protoplasts

Lactobacillus cells may be grown in MRS broth (Difco) at 37° C. for 3 hours to overnight. The cells may then be centrifuged at 2000×g for 30 minutes, and the resulting cell pellet washed and resuspended in a hypertonic solution (0.01 M Tris hydrochloride [pH 7.5], 0.3-0.5 M mannitol) that contains lysozyme (20 μg/ml) and incubated at room temperature for 5-15 minutes. The resulting protoplasts may be gently overlaid on plates with the appropriate regeneration media or formulated by mixing with suitable carriers such as yogurts or hypertonic solution having sucrose and appropriate buffers. Protoplasts must be maintained in the hypertonic solution, which may contain sucrose instead of mannitol, until they regenerate cell walls to prevent lysis due to osmotic pressure.

### EXAMPLE II

### Creation of a Lactobacillus Strain Having an Inducible Autolysin Gene

An expression cassette comprising an autolysing gene such as AcmA, holin or lysin as disclosed above may be operably linked to a lactose promoter such as the bacterial P_{lac} promoter or a pH dependent promoter. With respect to the Plac promoter, for example, this may be achieved by cloning the autolysing gene in pBluescript from Stratagene Cloning Systems (La Jolla, Calif.).

Targeted DNA sequences from the chromosomal DNA from lactobacillus, for example, may be obtained by generating a genomic library from the lactobacillus organism. pBluescript carrying the autolysing gene expression cassette may then be linearized by appropriate restriction enzyme and inserted in a number of genomic clones from the lactobacillus library. Preferably, clones to be used are selected to contain certain biochemical enzymes involved in the pathway for metabolizing certain nutrients or amino acids such as tryptophan, tyrosine, etc. and the insertion of pBluescript disrupts the particular enzyme in the particular metabolic pathway.

The resulting modified genomic DNA clone may then be transformed back into lactobacillus using any one of the transformation protocols discussed above. When the modified genomic DNA clone is in the cell, it may homologously recombine with the endogenous chromosomal DNA and resulting in the integration of the autolysing gene into the lactobacillus genome. Selection of mutants may be by antibiotic resistance conferred by the pBluescript plasmid or with the loss of the cells ability to grow with the nutrients whose metabolic pathway has been disrupted. It should be noted that mutant lactobacillus having the P_{lac} promoter driving the expression of an autolysing gene should be grown and propagated in lactose-free medium. The mutant lactobacillus may easily be grown in glucose containing medium.

In addition to autolysing gene, any number of genes may be introduced and integrated into the lactobacillus genome to create mutants for particular purposes. EXAMPLE III

### In Vitro Model for M-Cell Targeting with Bacteria Carrying GFP Expression Cassettes

To initially test the ability of recombinant LAB to target M-cells for delivery of the plasmid DNA, an in vitro model of intestinal cells having differentiated M-cells may be used, as described in Kerneis, S. et. al., “Conversion by Peyer's Patch Lymphocytes of Human Enterocytes into M-cells that Transport Bacteria,” Science, 277 (5328): 949 (1997), which is hereby incorporated by reference. Briefly, follicle-associated epithelium (FAE) and M-cells were established by cultivation of Peyer's patches lymphocytes with the differentiated human intestinal cell line Caco-2. 3×105 Caco-2 cells may be initially cultured overnight on the lower face of a 6.5 mm filter (3 micrometer pore Transwell filters, COSTAR, Cambridge, Mass.). The filters may then be transferred in the Transwell device with the epithelial cells facing the lower chamber of the cluster plates. Epithelial cells may then be cultured until they were fully differentiated (14 days). Lymphocytes may be isolated from PPs of BALB/c mice, dissociated, sorted by FACS using a monoclonal antibody to mouse B220 (CD45) and a monoclonal antibody to mouse CD3 T cells. After isolation, the lymphoid cells (106) may then be added in the upper chamber facing the basolateral side of the Caco-2 cells. After culturing for two days, the lymphocytes will most likely have accumulated in the intraepithelial pockets similar to that observed in M-cells in vivo, the Caco-2 cells will most likely have been converted to cells having M-cell phenotypes. Thus, this in vitro model may then be used to perform experiments to assess the conditions for targeting the recombinant LAB to the M-cells.

With this in vitro model, a culture of protoplasts as prepared using Example I or recombinant LAB as described above having targeting compound may then be incubated with the M-cell in vitro. The protoplasts or recombinant LAB may carry a plasmid with a CMV promoter, for example, operably linked with GFP. Expression of GFP in the M-cells may then be monitored using a fluorescent microscope.

Alternatively, a secreted alkaline phosphatase enzyme may be used as the reporter gene such as the commercially available pSEAP2-Basic vector from Clontech Laboratories (Palo Alto, Calif.). The secreted alkaline phosphatase enzyme may then be assayed from the culture media using the manufacturer's protocol. Furthermore, the pSEAP2 vector may be modified such that a targeted signal peptide sequence may be used for either apical or basolateral targeting of the alkaline phosphatase. Alkaline phosphatase activity may then be assayed either in the upper or lower chamber of the Transwell depending on whether the targeted secretion was apical or 10 basolateral.

It should be understood that the particular experiment is not limited to reporter genes but may also use any other proteins of interests together with ELISA or immunohistochemistry to determine the introduction of the DNA to the M-cells.

In yet another example, ever increasing amounts of plasmid DNA having the GFP gene may be incubated with the in vitro M-cell model and assayed for the uptake of naked plasmid DNA by the M-cell. Alternatively, lysates from the recombinant LAB or protoplasts may also be incubated with the in vitro model and assayed for the uptake of plasmid DNA using GFP.

### EXAMPLE IV

### In Vivo Assays for Secreted Alkaline Phosphatase

Based on the data accumulated on the concentration of bacteria, protoplasts or DNA needed for M-cells to take up the DNA, appropriate concentration of protoplasts or recombinant LAB may then be formulated as described and administered to an animal or human through oral ingestion. If secreted alkaline phosphatase were to be used, then serum from the animals or human may be collected and assayed for SEAP activity using Clontech's Great EscAPE™ SEAP Chemiluminescence Detection Kit (Cat. # K2041-1) and its protocol, which is incorporated herein by reference. The SEAP enzyme expressed from Clontech's pSEAP-2 vector is thermostable. Thus, to determine the level of SEAP activity as opposed to the endogenous alkaline phosphatase enzyme activity, the assay would require the deactivation of the endogenous alkaline phosphatase enzyme by heating the samples at 65° C. for thirty minutes before adding the chemiluminescence substrate.

Alternatively, if the protein to be expressed is insulin, then the recombinant LAB having the appropriate insulin expression cassette may be administered to appropriate animal models of the diabetes disease or to diabetic individuals. The effect of the treatment may then be monitored based on the level of glucose and insulin in the animal's or human's bloodstream using well known techniques in the art.

If the protein to be expressed is an antigen, then serum from the animal or human may then be collected and assayed for antibodies that would bind to the antigen that was introduced.

### EXAMPLE V

### High Copy Plasmids

To increase the chance of the plasmid DNA actually entering the cells in the epithelial lining, it is preferred that high copy plasmid be used to transform the bacteria. An example of high copy plasmid include plasmid with mutated origin of replication such as the pUC 18 vectors where repressors are more relaxed in binding to the origin of replication. Similar mutations may be made on the origin of replication of the LAB plasmid vectors to increase the copy number of the plasmid. Mutations may be induced my random mutagenesis using well-known techniques of PCR reactions. Primers flanking the origin of replication may be designed and a non-proofreading polymerase such as Taq polymerase may then used, preferably in manganese buffer instead of magnesium buffer. The mutated PCR fragments may then be substituted with the original origin and replication and transformed into the bacteria. A high selective pressure such as high antibiotic in the media may then be used to select for high copy LAB plasmid.

Alternatively, “runaway” plasmid may be used for achieving high copy plasmid number. “Runaway” plasmid is part of a class of versatile plasmids discovered and developed in E. coli. They are based on the IncFII plasmid, R1, in which an antisense RNA (CopA RNA) negatively controls the formation of a protein that is rate-limiting for replication. The copy number of the plasmid is determined by the balance between the rates of formation of CopA RNA and RepA MRNA. A small increase in the rate of formation of the latter drastically reduces the rate of formation of CopA RNA due to convergent transcription, which may lead to a total loss of copy number control (runaway replication), resulting in massive DNA amplification, and plasmid copy numbers up to 1000 per genome. Since this amplification occurs in the presence of protein synthesis, the protein that is encoded by a cloned gene can also be amplified, and may constitute 10-50% of the total protein. See e.g., Nordstrom, K and Uhlin, BE, “Runaway-replication plasmids as tools to produce large quantities of proteins from cloned genes in bacteria,” Biotechnology (N Y) 1992 June; 10(6):661-6.

Current available “runaway” plasmids may be modified to carry expression cassettes suitable for mammalian expression. The modified “runaway” plasmid may then be delivered to the gastrointestinal tract using non-pathogenic E. coli cells and lysing the cells according to methods described above. “Runaway” plasmids may also be developed for lactobacillus and lactococcus for used in the delivery of DNA.

### EXAMPLE VI

### Production of a Modified Lactococcus Organism

Selection of Bacteria and Cloning of the Plasmid DNA

The modified Lactococcus organism will be formed through the fusion of Lactococcus with a second bacteria that contains a recombinant plasmid. In this example, Lactococcus lactis (ATCC #7962) will be fused with E. coli HB101 (ATCC # 33694).

The E. coli HB 101 will contain a recombinant plasmid, pSYG3 that encodes GFPuv, which is a GFP variant that has been optimized for bacterial expression (Crameri, A., et al. “Improved green fluorescent protein by molecular evolution using DNA shuffling” Nat. Biotechnol. 14: 315-19 (1996)). GFPuv has been optimized for maximal fluorescence when excited by UV light (360-400 nm) and can be amplified from pBAD-GFPuv (Clontech, Palo Alto, Calif.) using the following primers: CAT GCA TGC CAT GGC TAG CM AGG AGA AGA AC and CCG GGT ACC GAG CTC GM TTC (SEQ. ID. NO 1)(Geoffroy, M., et al., “Use of green fluorescent protein to tag lactic acid bacterium strains under development as live vaccine vectors” Applied and Environmental Microbiology 66(1): 383-91 (2000)).

PSYG3 will be constructed from pUC19 and will include the origin of replication from pBR322, a kanamycin resistance gene, and a T7 promoter sequence that is operably linked to a nucleotide sequence encoding GFPuv fused with surface binding promoter regions. The surface binding promoter regions may be sequences for the signal peptide from the lactococcal Usp45 preprotein and for the cell wall anchor domain from the M6 preproprotein of Streptococcus pyogenes along with the necessary transcriptional terminators. The signal peptide sequence will be upstream from the GFPuv sequence while the cell wall anchor domain will be downstream from the GFPuv sequence. For details, see Deite, Y., et al., “Design of a protein-targeting system for lactic acid bacteria” Journal of Bacteriology 183(14): 4157-66 (2001). Cloning of the plasmid, transformation of the E. coli cells with the plasmid, and selection of colonies containing the plasmid will be accomplished according to procedures that are well known to one of ordinary skill in the art as set forth in references such as Sambrook, et al., Molecular Cloning: A Laboratory Manual, 3^{rd} edition (Cold Spring Harbor Press, Cold Spring Harbor, N.Y.) (2001) and Ausubel, et al., Current Protocols in Molecular Biology, (Wiley, New York) (2001).

Alternatively, the plasmid may also contain other DNA sequences, such as a sequence encoding the sigma 1 protein of reovirus operably linked to a T7 promoter in addition to surface binding promoter regions, such as those described above. Expression of such a protein would accomplish M-cell targeting.

Formation of Escherichia Coli and Lactococcus Protoplasts

Protoplasts of both bacterial strains may be formed using the following methods. Lactococcus lactis cells will be grown in MRS media (Difco) at 26 C until the exponential growth phase has been reached. E. coli HB 101 harboring pSYG3 will be grown in LB at 37 C for until the exponential growth phase has been reached. Then, chloramphenicol will be added to the E. coli culture and pSYG3 selectively amplified for 16 hours. After centrifugation of the cultures at 2000×g for 30 minutes, the resulting cell pellets will be washed and resuspended in a hypertonic solution (0.01 M Tris hydrochloride [pH 7.5], 0.3-0.5 M mannitol) that contains lysozyme (20 ug/ml) and incubated at room temperature for 5-15 minutes. An aliquot of the resulting protoplasts will be gently overlaid on plates with the appropriate regeneration media (MRS or LB) and colony formation observed to insure the protoplasts are able to regenerate cell walls. Protoplasts must be maintained in the hypertonic solution, which may contain sucrose instead of mannitol, until they regenerate cell walls, to prevent lysis due to osmotic pressure.

Fusion of E. coli and L. lactis Protoplasts

To fuse the protoplasts, 1×10⁹-10×10¹⁰ E. coli protoplasts in the hypertonic solution described above may be added to 0.5-1 ml of the L. lactis protoplasts 1×10⁹-10×10⁹ in the same hypertonic solution. 0.5 ml-1.5 ml of 20%-70% PVA or PEG will be added to the mixture, and the solution will be gently agitated to achieve thorough mixing. The mixture may be incubated for 1-30 minutes at room temperature, and protoplast aggregation and fusion monitored by phase-contrast microscopy. When cell growth reaches an exponential stage, the protoplasts will be washed three times and diluted in 3-7 ml of the hypertonic solution used above. A small amount of the resulting solution (0.5-2 ml) will be plated on MRS agar with kanamycin and incubated at 26 C.

The MRS agar will select for L. lactis and modified L. lactis, replica on a minimum medium and/or an ELISA test can be performed with antiserum against LAB. LAB strains identification will also be performed on the tomato agar plates. The kanamycin will select for bacteria containing pSYG3. Thus, the resulting colonies will be modified 1. acidophilus fusants harboring pSYG3. Alternatively, because GFP is also a reporter gene, colonies containing pSYG3 may be selected based on green fluorescence under ultraviolet light.

### EXAMPLE VII

### Characterization of the Phenotype of the Modified Lactococcus Organism

Various assays will be performed to confirm that the desired modified lactobacillus organism has been generated. Single colonies will be 1) picked from the selective plates described above, 2) grown in MRS broth, and 3) replated on MRS agar with kanamycin. Steps 1-3 will be repeated five times to obtain purified colonies.

Tests to determine the physiological properties of the modified lactobacillus organism will be performed according to the instruction manual from the API ZYM and API 20A biochemical test systems. The characteristics of the parent bacteria are set forth in Holt, et al., Bergey's Manual of Determinative Bacteriology gth ed. (Williams & Wilkins, Baltimore, Md.) (1994), which is a comprehensive guide that allows identification of bacteria that have been described and cultured.

Based on the selective pressures described above, the modified Lactococcus organism should have a phenotype corresponding to that of the genus Lactococcus. Therefore, the cells should be spherical and Gram positive. In liquid media, the cells will occur in pairs or in short chains. They should require a complex media for growth, and their metabolism should be fermentative, producing L(+)-lactic acid without gas. In addition, the cells should be catalase negative and oxidase negative.

The modified Lactococuss organism should not have a phenotype corresponding to the genus Escherichia. Some of the above tests for lactobacillus will also show that the modified lactobacillus organism is not Escherichia, as Escherichia cells reduce nitrates, are gram negative, and are catalase positive.

### EXAMPLE VIII

### Characterization of the Genotype of the Modified Lactococcus Organism

Southern blots will be performed to determine whether the modified Lactococcus organism has the expected genotype. Chromosomal DNA will be extracted according to standard procedures. See Saito and Miura. Plasmid DNA preparation and Southern, hybridization will be performed as described in Sambrook, Molecular Cloning: A Laboratory Manual.

Chromosomal DNA from the parent bacteria and plasmid DNA will be used as probes. Low homology would be observed if Lactococcus lactis chromosomal DNA were probed with E. coli chromosomal DNA or if E. coli were probed with L. lactis DNA. In contrast, the L. lactis and E. coli chromosomal DNA probes will share 50% or greater homology with the modified Lactococcus chromosomal DNA, as the fusant should contain chromosomal DNA from both of the parent bacteria. One of skill of the art would also appreciate that if the two parents are more closely related and therefore have highly homologous chromosomal DNA, as may occur in some embodiments of this invention, the difference in the degree of hybridization that occurs between the chromosomal DNA of the two parents and the degree of hybridization that occurs between the parent and fusant chromosomal DNA will be less dramatic than that described in this example. In such cases, one may rely more heavily on identification of the plasmid DNA through Southern hybridization to characterize the genotype of the fusant.

### EXAMPLE IX

### Assays to Determine Expression of Antigen in the Modified Lactococcus Organism Ex Vivo

Detection of GFP Fluorescence

Expression of GFP fluorescence in the modified Lactococcus organism may be examined in several ways, according to known procedures. As noted above, plates with the modified Lactococcus organism may be photographed under UV illumination to identify colonies that are expressing GFP. In addition, GFP production in modified Lactococcus cells suspended in PBS may be observed using epifluorescence microscopy. Photographs of such observations using appropriate film may be taken. Finally, GFP expression may be measured by preparing modified Lactococcus cell lysates and assaying for fluorescence using a fluorimeter.

Western Blots Performed on Total Protein Extracts and Cell Fractions to Localize GFP Expression

Western blots of total protein extracts and various fractions of the cell will be performed to test for expression of GFPuv and to show that GFP is being targeted to the cell membrane. Total protein extracts will be prepared according to well-known procedures set forth in references such as Ausubel, et al., Current Protocols in Molecular Biology. Cell fractionation will be performed according to the method outlined in Piard, J.-C., et al. “Cell wall anchoring of the Streptococcus pyogenes M6 protein in various lactic acid bacteria.” Briefly, 2 nil of exponential-phase culture may be microcentrifuged for 5 minutes at 4 C at 4,300 g. The resulting cell pellet and supernatant will be separated and concentrated. Proteins in the supernatant will be precipitated using trichloroacetic acid (TCA). The cell pellet will be resuspended in TES, treated with lysozyme, and the resulting protoplasts centrifuged at low speed. The supernatant will contain proteins released from the cell wall, which will be precipitated using TCA. Proteins will then be extracted from the protoplast pellet as described in Dieye, Y., et al. “Design of protein-targeting system for lactic acid bacteria” Journal of Bacteriology 183(14): 4157-66.

Total protein and cell fraction samples may then be analyzed by Western blot using rabbit GFP antiserum (Invitrogen) as the primary antibody and horseradish peroxidase conjugated anti rabbit antisera (Sigma) as the secondary antibody and for detection. A known amount of recombinant GFPuv (Clontech) will be run as a control. The amount of GFPuv on the Western blots may be estimated by scanning them and comparing the signals from the control and experimental lanes. Western blotting is described in detail in Sambrook, et al., Molecular Cloning: A Laboratory Manual.

### EXAMPLE X

### Delivering HBSAG Antigen And IL-2 Gene

HBV surface antigen genes Pre-S2 and S will be obtained by PCR amplification from plasmid pEco63 (ATCC31518). Mouse IL-2 gene fragment will be obtained by PCR from plasmid pMUT-1 (ATCC37553). Both genes will be placed under Lac-Z promoter in fusion or under a separate T7 promoter in pUC18. The genes may also be cloned in a shuttle vector. Plasmid containing only pre-S2/S gene is named pPS2S. The plasmid with both pre-S2/S and IL-2 genes is named pPS2S/IL2. (Chow et al. J Vir. January 1997: 169-178). The two genes may also be cloned into another shuttle vector in a fusion or under a separate promoter. The DNA will be transformed into E. coli DH5 a and or HB 101. Plasmid DNA will then be amplified in E. coli cultures. Exponentially grown E. coli will be protoplasted as described above and fused with Lactococcus lactis. Fusants will be selectively grown on LAB MRC plate and tomato juice plate and or a synthetic medium (Broach et al. Gene. 8(1979)121-133.). Selection will be made for the expression plasmid via Kanamycin along with a transgene product assay, as following.

The HBsAg protein in the fusant medium broth or cell pellets will be assayed by the AUSZYME Monoclonal antibody kit (Abbott Lab). The intracellular protein should be released by a Ten-Brock ground bead homogenizer. Membrane bound proteins should be released by treatment with Triton X-100. Production of the antigen should be found up to 3% of total cellular protein. IL-2 activity will then be tested by a proliferated assay (Chow et al) and a ELISA using anti-IL-2 antibody.(Pharmigen).

BALB/c and C57b1/6 mice will be immunized with 1-10×10⁹ cfu of LAB of up to 3 doses. Serum will be collected by tail bleeding beginning from day 2. HbsAg antibodies will determined using serological methods known to those skilled in the art or as described previously herein.

### EXAMPLE XI

Construction of pYD1-Based Plasmids

pYD1 is a galactose-inducible expression vector purchased from Invitrogen, which directs expression of proteins on the yeast cell wall. The antigens of interest, VP7, HA and NA were PCR amplified using the primers listed in Table 1. The resulting PCR products were cloned into either the BamHI/EcoRI (VP7) or the BamHI/XbaI (NA and HA) sites of pYD1.

### EXAMPLE XII

### Construction of pGPD-DSPLY and it's Derivatives

pGPD-DSPLY functions as a target vector for constitutive expression of a number of proteins displayed on the cell wall. Names and sequences of PCR primers used to construct PGPD-DSPLY and it's derivatives are listed in Table 1. pGPD-DSPLY contains sequences encoding the leader sequence of yeast α-mating factor and the cell-wall anchoring domain (C-terminal 350 amino acids) of Saccharomycse cerevisiae α-agglutinin. First, sequences encoding the α-leader peptide followed by two amino acid spacers (Gly and Ala) were PCR amplified from the yeast chromosome (strain S288C) using primers BamLALPHAfwd and EcoLALPHArev and cloned into BamHI and EcoRI sites of p426GPD (described in Mumberg et al., 1995, Yeast vectors for the controlled expression of heterologous proteins in different genetic backgrounds, Gene 156: 119-122) to construct pSecY. Next, sequences encoding the cell-wall anchoring domain of α-agglutinin was PCR amplified from yeast chromosomal DNA (strain S288C), using the oligonucleotides Agglfwd and Aggirev, and cloned into the ClaI/XhoI sites of p426GPD to obtain pGPDAnch. PGPD-DSPLY was constructed by subcloning an EcoRI/XhoI fragment containing α-agglutinin sequences into the same sites of pSecY.

Vectors for surface display of antigens NA, VP7 (pNADSPLY, pVP7DSPLY) were constructed as follows: NA and VP7 encoding sequences were PCR amplified from a cloned copy of these gene using primer pairs NAnewfwd/NAnewrev and VP7newfwdNP7newrev, respectively, and cloned upstream of α-agglutinin sequences into the EcoRI/HindIII sites of pGPDAnch to obtain pNAAnch and pVP7Anch. Next, an EcoRI/XhoI fragment from pNAAnch and pVP7Anch were subcloned into the same sites of pSecY to obtain pNADSPLY and pVP7DSPLY, respectively. To verify correct positioning of antigens to the cells wall, pGFPDSPLY was constructed basically as described above; GFP encoding sequences were PCR amplified from plasmid pQB125-fPA (Qbiogene) using primers sgGFPfwd and sgGFPrev.

Construction of an HA surface display vector pHADSPLY was performed by cloning PCR-amplified HA sequences into the EcoRI/HindIII sites of PGPDDSPLY. Due to the presence of an EcoRI site within HA ecoding sequences, a sticky end PCR strategy was used (Zheng, G., Sticky-end PCR: new method for subcloning. 1998, Biotechniques, 25: 206-208) to facilitate the cloning. First, two separate HA amplification reactions were performed using primer pairs HAfwd1/Hanewrev and HAfwd2/HAnewrev. After digestion with DpnI (to remove background plasmid) and HindIII, equal molar amounts of the two PCR products were mixed, heat denatured, allowed to cool to room temperature, and cloned into the EcoRI/HindIII sites of pGPDDSPLY.

In order to facilitate immunological detection of the antigens, sequences encoding various epitope tags (His₆ and HA) were cloned into the EcoRI sites of pNADSPLY and pVP7DSPLY vectors which positions the tag in between the antigen-encoding, and the cell-wall anchoring sequences. The oligonucleotides used for these constructions are listed in Table 1.

### EXAMPLE XIII

### Preparation of Lactobacillus Surface Display Vectors

Genes expressing antigens of interest were cloned into SfiI/AscI sites of the surface display vector pSC111AE. As a result of this construction, VP7, HA, NA and GFP are fused N-terminally to the secretion signal of the amylase gene and C-terminally to the cell-wall anchoring domain of the prtp protease. The expression of the fusion proteins is driven by the constitutively active Xyl promoter. The sequences of oligonucleotides used for PCR amplification of the various antigens are shown in Table 1.

TABLE 1

SEQ ID NO for oligonucleotides used for construction of surface display
expression vectors

| | | | |
|---|---|---|---|
| | | | Target |
| SEQ ID | | | vector or |
| NO. | Oligonucleotide | Sequence | purpose |

| | | | |
|---|---|---|---|
| 2 | VP7-1 | 5′-CGGGATCCGGTGGCCAGAACTATGGACTTAATATAC-3′ | pYD-1 |
| 3 | VP7-2 | 5′-CCGGAATTCTTAATTTATCCCATCAACGAC-3′ | pYD-1 |
| 4 | HA-1 | 5′-CGGGATCCGGTGGTGGTGACACAATATTATAGGC-3′ | pYD-1 |
| 5 | HA-2 | 5′-CCGGAATTCTTAGATGCATATTCTGCAC - 3′ | pYD-1 |
| 6 | NA-1 | 5′-CGGGATCCGGTGGTGGTCATTCAATTCAAACTGG-3′ | pYD-1 |
| 7 | NA-2 | 5′-CCGGAATTCTTACTTGTCAATGGTGAA - 3′ | pYD-1 |
| 8 | BamLALPHAfwd | 5′-CCGGATCCATGAGATTTCCTTCAATTTTTAC-3′ | p426GPD |
| 9 | EcoLALPHArev | 5′-GCGAATTCAGCACCTCTTTTATCCAAAGATACC-3′ | p426GPD |
| 10 | Agglfwd | 5′-CCATCGATGGTTCTGCTAGCGCCAAAAGCTC-3′ | p426GPD |
| 11 | Agglrev | 5′-CAGCTCGAGTTAGAATAGCAGGTACGAC-3′ | p426GPD |
| 12 | HAfwd1 | 5′-AATTCGACACAATATGTATAGGCTAC-3′ | pGPDAnch |
| 13 | HAfwd2 | 5′-CGACACAATA TGTATAGGCTAC-3′ | pGPDAnch |
| 14 | HAnewrev | 5′-ACCAAGCTTGATGCATATTCTGCAC-3′ | pGPDAnch |
| 15 | NAnewfwd | 5′-CGGAATTCCATTCAATTCAAACTGGAAC-3′ | pGPDAnch |
| 16 | NAnewrev | 5′-ACCAAGCTTCTTGTCAATGGTGAATGG-3′ | pGPDAnch |
| 17 | VP7newfwd | 5′-CGGAATTCCAGAACTATGGACTTAATATAC-3′ | pGPDAnch |
| 18 | VP7newrev | 5′-ACCAAGCTTATTTATCCCATCAACGAC-3′ | pGPDAnch |
| 19 | sgGFPfwd | 5′-CGGAATTCATGGCTAGCAAAGGAGAAG-3′ | pGPDAnch |
| 20 | sgGFPrev | 5′-GGAAGCTTATCGATGTTGTACAGTTC-3′ | pGPDAnch |
| 21 | HAECOfwd | 5′-AATTTTACCCATACGACGTCCCAGATTACGCTGGTGCCG-3′ | epitope TAG |
| 22 | HAECOrev | 5′-AATTCGGCACCAGCGTAAACTGGGACGTCGTATGGGTAA-3′ | epitope TAG |
| 23 | HISECOfwd | 5′-AATTTCATCACCATCACCATCACGGTGCCG-3′ | epitope TAG |
| 24 | HISECOrev | 5′-AATTCGGCACCGTGATGGTGATGGTGATGA-3′ | epitope TAG |
| 25 | GfpSfilForward | 5′-TAGGCCCAGCCGGCCGCCGCTAGCAAAGGAGAAGAACTCTTCACTGG-3′ | pSc111AE |
| 26 | GFPAsclReverse | 5′-AAGGCGCGCCATCGATGTTGTACAGTTCATC-3′ | pSC111AE |
| 27 | Vp7SfilForward | 5′-TAGGCCCAGCCGGCCGCCCAGAACTATGGACTTAATATAC-3′pSC111AE | |
| 28 | Vp7AsclReverse | 5′-AAGGCGCGCCATTTATCCCATCAACGAC-3′ | pSC111AE |
| 29 | HASfilForward | 5′-TAGGCCCAGCCGGCCGCCGACACAATATGTATAGGCTAC-3′ | pSC111AE |
| 30 | HAAsclReverse | 5′-TAGGCCCAGCCGGCCGCCCATTCAATTCAAACTGGAAGTC-3′ | pSC111AE |
| 31 | NASfilForward | 5′-TAGGCCCAGCCGGCCGCCCATTCAATTCAAACTGGAAGTC-3′ | pSC111AE |
| 32 | NAAsclReverse | 5′-AAGGCGCGCCCTTGTCAATGGTGAATGG-3′ | pSC111AE |

### EXAMPLE XIV

### Expression of Proteins on Yeast Cell Surface

pYD1-based expression-EBY 100 yeast transformed with pYD1 or pYD1-based expression vectors were grown overnight at 30° C. in YNB-CAA medium containing 2% glucose. Cells were harvested by centrifugation and resuspended in YNB-CAA medium containing 2% galactose to an OD₆₀₀ of 0.5˜1. Cells were grown at 20˜25° C., and samples were harvested at regular time intervals to analyze for expression by immunofluorescent staining.

pGPD-DSPLY-based expression- W303-1A cells transformed with pGDP-DSPLY or it's derivatives were grown to mid-log phase at 30° C. in Synthetic drop out medium without uracil. Cell were harvested and analyzed for protein expression as described below.

### EXAMPLE XV

### Detection of Antigens on Yeast Cell Surface

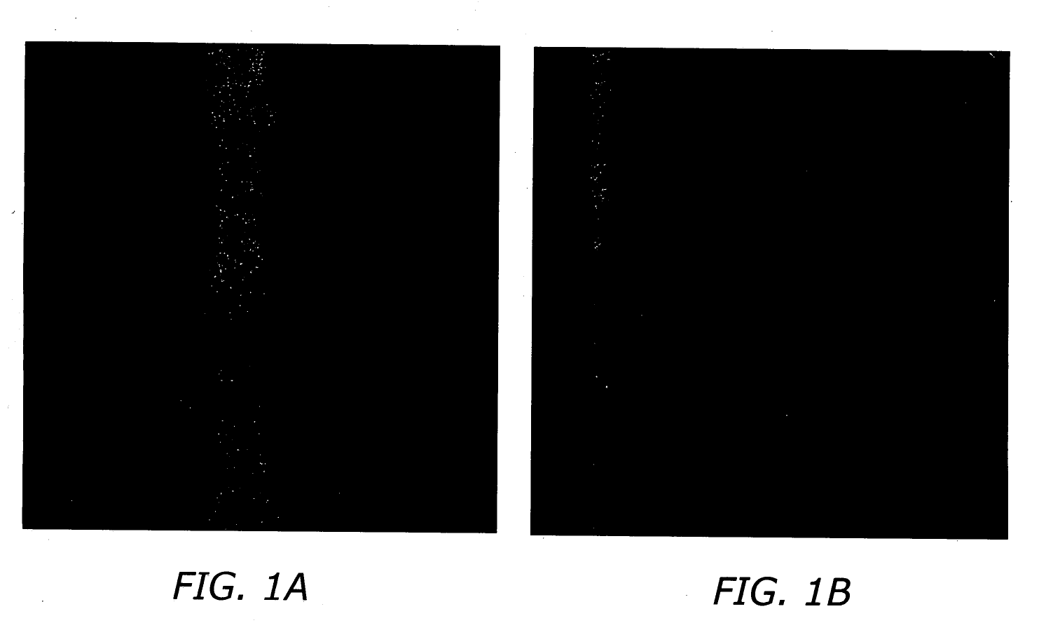

Detection of antigens on yeast cell surface was accomplished by immunofluorescence labeling of whole cells followed by confocal microscopy. An exponentially growing culture of yeast was fixed by addition of 1/10^{th} volume of formaldehye to the culture medium, with continued incubation of the shaking culture for 1 Hour. The fixed cells were washed with PBS three times and incubated with a monoclonal anti-GFP antibody for 1.5 hrs at room temperature (RT). After washing with PBS, the cells were incubated for 1 hr at RT with the secondary antibody conjugated with Rhodamine. Cells were washed with PBS, mounted on a microscope slide and visualized with confocal microscopy. As shown in FIG. 1, GFP was expressed on the surface of yeast cells as indicated by the pattern of the cellular distribution of GFP-associated fluorescence. In addition, a similar pattern of GFP distribution was detected by immunofluorescence analysis of yeast cells expressing surface-displayed GFP.

### EXAMPLE XVI

### Protocol for Immunization of Animals with Recombinant Yeast

Six weeks old female Balb/c mice were inoculated by oral, intranasal or subcutaneous routes with yeast displaying VP7, HA or NA on the cell surface. Booster inoculations were performed every two weeks. Mice were inoculated with either yeast expressing surface-displayed antigen or yeast containing empty vector. Three different routes of inoculation were used: oral, intranasal or subcutaneous. The number of mice used for each experiment is outlined in Table 2. Blood samples were collected before the first vaccination and every two weeks there after. Mice were sacrificed after 8-weeks, and trachea, lung and intestine washings were collected. The presence of antigen-specific IgG and IgA antibodies in the blood and tissue samples were detected by ELISA.

A. Vaccine Preparation

For the galactose-inducible expression (pYD1), yeast cells expressing virus antigens VP7, HA or NA, and cells containing empty vector were grown in YNB-CAA medium and induced for expression with 2% galactose. For constitutive expression (pGPD-DSPLY), yeast cells were grown to mid-log phase in synthetic defined (SD) dropout media without uracil. Cells were harvested at mid-log phase, washed with and resuspended in PBS to a concentration of 5×10⁹/ml.

B. Vaccination

Oral: 0.1 ml (5×10⁸)/mice
Intra-nasal: 0.02 ml (1×10⁸)/mice
Subcutaneous: 0.1 ml (5×10⁸) mixed with 0.1 ml adjuvant/mice (complete Freund's adjuvant for the first Subcutaneous inoculation, incomplete Freund's adjuvant for booster). The first inoculation was done on week zero. Booster inoculations were done at weeks two, four and six with the same amount of cells.

### EXAMPLE XVII

### Measurement of Antibody Response

Blood samples (˜0.1 ml) were taken from the eye bowl. Serum were separated by centrifugation, and stored at −20° C. The Lung and intestines were separated from the sacrificed animal and washed with PBS. The tissue washings were collected into Eppendorf tubes and centrifuged. The supernatants were stored at −20° C.

The samples were tested by ELISA for the presence of antigen-specific antibodies. The Viral antigens, VP7, HA or NA were coated on 96 well plates. After blocking of non-specific binding sites, samples of sera, lung or intestine washings were diluted with PBS and added to each well. Horseradish peroxidase-labeled secondary antibodies (anti-IgG or anti-IgA) were used to detect antibody-antigen complexes.

Tables 3, 4, 5 and 6 below show the raw data from each vaccination protocol. Table 3 shows serum antibody titer for yeast Flu vaccine using pGPD Table 4 shows serum antibody titer for yeast rotavirus vaccine using pGPD Table 5 shows serum antibody titer for yeast Flu vaccine using pYD1 Table 5. Serum antibody titer for yeast Flu vaccine using pYD and Table 6 shows serum antibody titer for yeast rotavirus Vaccine for pYD1

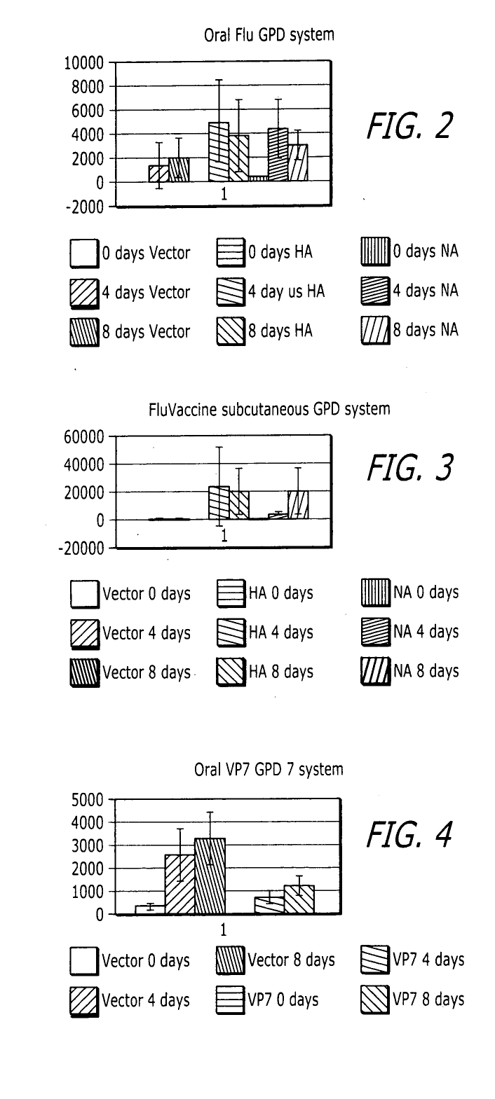

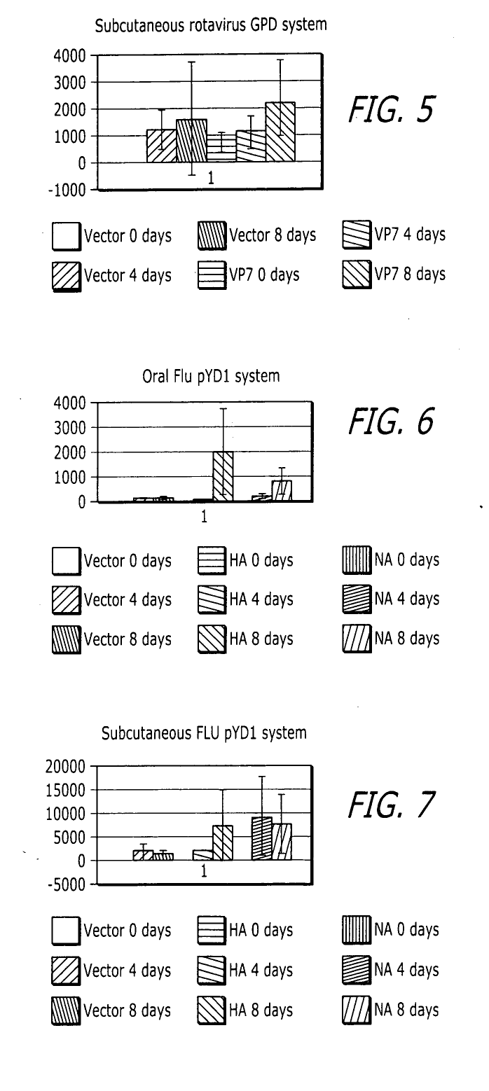

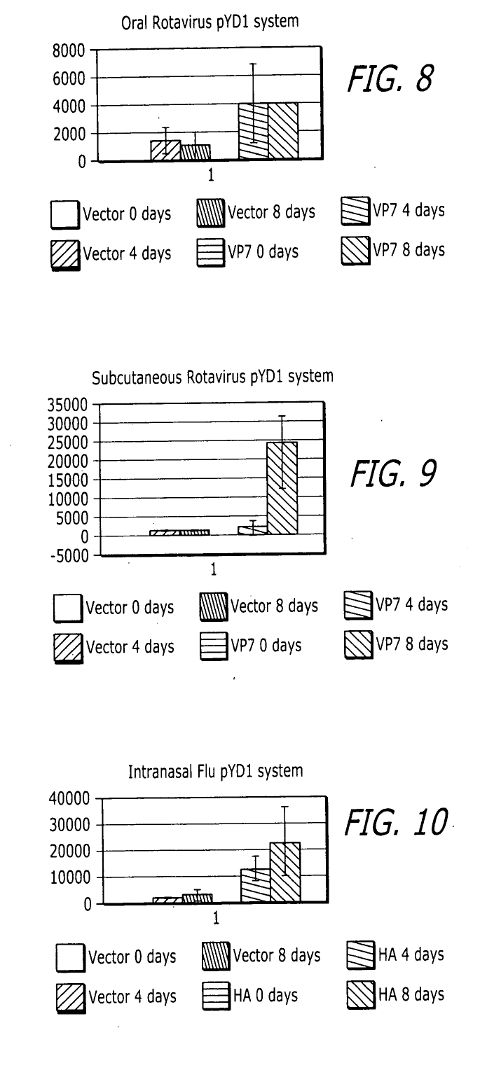

FIGS. 2-10 graphically depict the data presented in Tables 3-6. As can been seen, when compared to the plasmid controls, each immunogenic composition of the present invention successfully elicited an immune response in the test animal.

TABLE 2
Number of animals in each experimental group

| | Vaccine | | | | | | | |
|---|---|---|---|---|---|---|---|---|
| | A | A1 | A2 | A3 | B | B1 | B2 | B3 |
| | control | VP7 | HA | NA | control | VP7 | HA | NA |
| Oral | 4 | 4 | 4 | 4 | 4 | 4 | 4 | 4 |
| Intra-nasal | 4 | 4 | 4 | 4 | 4 | 4 | 4 | 4 |
| subcuta- | 4 | 4 | 4 | 4 | 4 | 4 | 4 | 4 |
| neous | | | | | | | | |

Note:
A, pYD1 system;
B, pGPD-DSPLY system.

TABLE 3
Serum antibody titer for yeast Flu vaccine using pGPD

| | | Vaccine | | | | | | | | |
|---|---|---|---|---|---|---|---|---|---|---|
| | | pGPD | | | pGPD-HA | | | PGPD-NA | | |
| weeks | | 0 | 4 | 8 | 0 | 4 | 8 | 0 | 4 | 8 |
| Oral | 1 | 0 | 0 | 2000 | 0 | 2000 | 8000 | 500 | 2000 | 2000 |
| | 2 | 0 | 2000 | 2000 | 0 | 8000 | 2000 | 500 | 4000 | 2000 |
| | 3 | 0 | 4000 | 4000 | 0 | 8000 | 4000 | 500 | 8000 | 4000 |
| | 4 | 0 | 0 | 0 | 0 | 2000 | 1000 | 500 | 4000 | 4000 |
| | Mean | <500 | 1500 | 2000 | <500 | 5000 | 3750 | 500 | 4500 | 3000 |
| | SD | 0 | 1915 | 1633 | 0 | 3464 | 3096 | 0 | 2517 | 1155 |
| SQ | 1 | 500 | 2000 | 2000 | 500 | 4000 | N/A | 500 | 4000 | N/A |
| | 2 | 250 | N/A | N/A | 250 | 64000 | N/A | 1000 | 4000 | 32000 |
| | 3 | 500 | 1000 | 500 | 250 | 16000 | 32000 | 500 | N/A | N/A |
| | 4 | 500 | 1000 | 1000 | 500 | 8000 | 8000 | 1000 | 2000 | 8000 |
| | Mean | 438 | 1333 | 1167 | 375 | 23000 | 20000 | 750 | 3333 | 20000 |
| | SD | 125 | 577 | 764 | 144 | 27785 | 16971 | 289 | 1155 | 16971 |

SQ = Subcutaneous

TABLE 4
Serum antibody titer for yeast rotavirus vaccine using pGPD

| | | Vaccine | | | | | |
|---|---|---|---|---|---|---|---|
| | | pGPD | | | pGPD-VP7 | | |
| weeks | | 0 | 4 | 8 | 0 | 4 | 8 |
| Oral | 1 | 500 | 2000 | 4000 | 0 | 500 | 1000 |
| | 2 | 250 | 2000 | 2000 | 0 | 1000 | 2000 |
| | 3 | 250 | 4000 | 4000 | 0 | 500 | 1000 |
| | 4 | N/A | N/A | N/A | 0 | 1000 | 1000 |
| | Mean | 333 | 2667 | 3333 | <500 | 750 | 1250 |
| | SD | 144 | 1155 | 1155 | 0 | 289 | 500 |
| SQ | 1 | 0 | 2000 | 250 | 500 | 1000 | 4000 |
| | 2 | 0 | N/A | N/A | 1000 | 2000 | 2000 |
| | 3 | 0 | 1000 | 4000 | 250 | 500 | 2000 |
| | 4 | 0 | 500 | 500 | 1000 | 1000 | 1000 |
| | Mean | <500 | 1167 | 1583 | 688 | 1125 | 2250 |
| | SD | 0 | 764 | 2097 | 375 | 629 | 1258 |

N/A means Not Available Serum antibody titer for yeast Flu vaccine

TABLE 5
Serum antibody titer for yeast Flu vaccine using pYD1

| | | Vaccine | | | | | | | | |
|---|---|---|---|---|---|---|---|---|---|---|
| | | pYD1 | | | pYD1-HA | | | pYD1-NA | | |
| weeks | | 0 | 4 | 8 | 0 | 4 | 8 | 0 | 4 | 8 |
| Oral | 1 | 0 | 1000 | 2000 | 0 | N/A | N/A | 0 | 2000 | 16000 |
| | 2 | 0 | 500 | 1000 | 0 | 500 | 32000 | 0 | 500 | 4000 |
| | 3 | 0 | 1000 | 1000 | 0 | N/A | N/A | 0 | 2000 | 8000 |
| | 4 | 0 | 1000 | 500 | 0 | 500 | 8000 | 0 | 2000 | 4000 |
| | Mean | 0 | 875 | 1125 | 0 | 500 | 20000 | 0 | 1625 | 8000 |
| | SD | 0 | 250 | 629 | 0 | 0 | 16970 | 0 | 750 | 5656 |
| IN | 1 | 0 | 2000 | 500 | 0 | 16000 | 8000 | 0 | 1000 | 8000 |
| | 2 | 0 | 2000 | 4000 | 0 | 8000 | 32000 | 0 | N/A | N/A |
| | 3 | 0 | 500 | 4000 | 0 | 16000 | 32000 | 0 | N/A | N/A |
| | 4 | 0 | 2000 | N/A | 0 | N/A | N/A | 0 | N/A | N/A |
| | Mean | 0 | 1625 | 2833 | 0 | 13333 | 24000 | 0 | 1000 | 8000 |
| | SD | 0 | 750 | 2020 | 0 | 4618 | 13856 | 0 | N/A | N/A |
| SQ | 1 | 0 | 2000 | 500 | 0 | 2000 | 16000 | 0 | 2000 | 2000 |
| | 2 | 0 | 4000 | 2000 | 0 | 2000 | 2000 | 0 | 16000 | 16000 |
| | 3 | 0 | 1000 | 1000 | 0 | 2000 | 4000 | 0 | 16000 | 4000 |
| | 4 | 0 | 250 | 2000 | 0 | 2000 | N/A | 0 | 2000 | 8000 |
| | Mean | 0 | 1812 | 1375 | 0 | 2000 | 7333 | 0 | 9000 | 7500 |
| | SD | 0 | 1625 | 750 | 0 | 0 | 7572 | 0 | 8082 | 6191 |

SQ = Subcutaneous

TABLE 6
Serum antibody titer for yeast rotavirus vaccine for pYD1

| | | Vaccine | | | | | |
|---|---|---|---|---|---|---|---|
| | | pYD1 | | | pYD1-VP7 | | |
| weeks | | 0 | 4 | 8 | 0 | 4 | 8 |
| Oral | 1 | 0 | 2000 | 2000 | 0 | 4000 | 4000 |
| | 2 | 0 | 0 | 2000 | 0 | 2000 | 4000 |
| | 3 | 0 | 2000 | 0 | 0 | 2000 | 4000 |
| | 4 | 0 | 2000 | 0 | 0 | 8000 | |
| | Mean | 0 | 1750 | 2000 | 0 | 3500 | 4000 |
| | SD | | | | | | |
| IN | 1 | | | | 0 | 1000 | 2000 |
| | 2 | | | | 0 | 500 | 1000 |
| | 3 | | | | 0 | 500 | 1000 |
| | 4 | | | | 0 | 1000 | 4000 |
| | Mean | | | | 0 | 750 | 2000 |
| | SD | | | | | | |
| SQ | 1 | 0 | 500 | 1000 | 0 | 500 | 32000 |
| | 2 | 0 | 1000 | 1000 | 0 | 1000 | 16000 |
| | 3 | 0 | 500 | 500 | 0 | 4000 | 16000 |
| | 4 | 0 | 1000 | | 0 | | |
| | Mean | 0 | 688 | 750 | 0 | 1833 | 21333 |
| | | | | 64 | | | |
| | SD | | | | | | |

### Administration and Delivery

The modified microflora of the present invention may be delivered to the intestinal mucosa for the delivery of antigens and heterologous nucleic acids to animals in need thereof. The immunogenic compositions and gene delivery compositions of the present invention can be compounded with a wide range of pharmaceutically acceptable excipients. Pharmaceutically acceptable carriers for therapeutic use are well known in the pharmaceutical art, and are described, for example, in Remington's Pharmaceutical Sciences (A. P. Gennaro, ed.; Mack, 1985). One administration regimen comprises one or more “initial” administrations; in days 1 to 6, followed by one or more booster administrations in days 12 to 24, and optionally one or more further booster administrations in days 30 to 36. A single initial administration, followed by a single booster administration, within this time period, will generally be sufficient.

Culture of the modified microflora can be formulated in a manner known per se, such as for the formulation or preparations of live bacteria for oral administration to animals such as preparations for the administration of probiotics, e.g. for the treatment of gastrointestinal disorders. The preparation may also be in a form suitable for administration into the stomach or intestinal mucosa through ingestion or via a tube or catheter. The culture of modified microflora according to the invention may be in a form suitable for oral administration, which may be a solid, semi-solid or liquid form, including but not limited to solutions and/or suspensions of the bacteria. The composition may also be in the form of an enteric coated composition. Suitable encapsulating compounds include but are not limited to chitosan, maltodextrin, lipids and oligo- and polysaccharides. Such encapsulation may also improve the shelf-life of the bacterial culture. In addition, if desired; the present invention may also be formulated and used as suppositories for rectal or vaginal administration; aerosols or insufflations for intratracheobronchial administration; and the like. Preparations of such formulations are well known to those skilled in the pharmaceutical arts. The dosage and method of administration can be tailored to achieve optimal efficacy and will depend on factors that those skilled in the medical arts will recognize.

The culture of modified microflora may also be prepared in the form of a powder, such as a freeze dried powder that is reconstituted before use, e.g., using a suitable liquid; or in the form of a solid or liquid preparation that is mixed with solid, semi-solid or liquid food prior to administration. It may also be in the form of a fermented product, such as yogurt or cheese. As such, the culture of modified microflora may contain one or more pharmaceutically acceptable carriers/exipientia, such as water. The culture of modified microflora may also contain one or more adjuvants, including immune adjuvant, suitable for oral administration, as long as these are compatible with the bacterial or yeast host and do not interfere with its desired immunogenic properties. For example, sterile saline or phosphate-buffered saline at physiological pH may be used. Preservatives, stabilizers, dyes, and even flavoring agents may be provided in the pharmaceutical composition. For example, sodium benzoate, sorbic acid, and esters of p-hydroxybenzoic acid may be added as preservatives. Antioxidants and suspending agents may also be used.

For administration, the preferred route would be oral ingestion of the bacterial culture. Preferably, the formulation of a biological composition involves the fermentation of the modified microfloral strains within dairy products, i.e., in the preparation of yogurt and administration is to be oral, such as by the ingestion of yogurt on a daily basis. Hence, in another embodiment the present invention is directed to a novel pharmaceutical composition that includes the modified microflora encoding an effective amount of a DNA, cDNA, RNA or protein for the treatment and/or prevention of disease, and a biologically acceptable carrier, such as yogurt. With respect to lysozyme treated protoplasts, the protoplasts culture may also be generally mixed, prior to oral ingestion, with a non-toxic, pharmaceutically acceptable carrier substance, such as yogurt.

The effective amount of the modified microflora to be given to a particular patient will depend on a variety of factors, several of which will be different from patient to patient. A competent clinician will be able to determine an effective amount of a antigenic or therapeutic composition to administer to a patient to elicit an appropriate immune or therapeutic response. Dosage of the composition will depend on the type of treatment, route of administration, the nature of the antigens or therapeutics, calculated absorption rates for the therapeutics, etc. Utilizing LD₅₀ animal data, and other information available for ingestion and or absorption via the intestinal mucosa, a clinician can determine the maximum safe dose for an individual, depending on the route of administration. Utilizing ordinary skill, the competent clinician will be able to optimize the dosage of a particular therapeutic composition in the course of routine clinical trials.

All of the described methods for delivery and lysis in the gastrointestinal tract may also be similarly applied to E. coli bacteria, which are also naturally part of the body's flora. Non-pathogenic strain of E. coli may be selected or isolated from the intestinal mucosa. Expression plasmids and vectors and their transformation in E. coli are so well characterized in the prior art that the modifications described above may be achieved by routine recombinant technology.

Once properly compounded in accordance with the teachings of the present invention, the microflora compositions of the present invention can be used to elicit immune responses and provide heterologous nucleic acids to the intestinal mucosa of a wide range of animals including, but not limited to primates, goats, cattle, horses, birds, fish, pigs, rats, mice cats and dogs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for inducing an immune response in an animal comprising:
providing an immunogenic composition formulated for oral administration to said animal wherein said immunogenic composition comprises a microflora organism having an expression vector wherein said expression vector comprises a heterologous nucleic acid that encodes for an antigen.

2. The method for inducing an immune response in an animal according to claim 1 wherein said microflora organism is a yeast or bacteria.

3. The method for inducing an immune response in an animal according to claim 1 wherein said antigen is selected from the group consisting of tumors, bacteria, viruses, parasites, and fungi.

4. The method for inducing an immune response in an animal according to claim 3 wherein said viruses are selected from the group consisting of influenza, hepatitis, HIV, and rotavirus.

5. The method for inducing an immune response in an animal according to claim 2 wherein said yeast in is selected from the group consisting of Saccharomyces cerevisiae, S. exiquus, S. telluris, S. dairensis., S. servazzii, S. unisporus, and S. kluyveri.

6. The method for inducing an immune response in an animal according to claim 2 wherein said bacteria in is selected from the group consisting of Bifidobacterium sp, Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

7. The method for inducing an immune response in an animal according to claim 1 wherein said oral formulation is selected from the group consisting of powder, a freeze dried powder a liquid preparation, a semi-solid, yogurt milk and cheese.

8. A method for inducing an immune response in an animal comprising:
providing an oral formulation of transformed yeast wherein said yeast comprise a heterologous nucleic acid encoding for an antigen where in said antigen is expressed on the surface of said yeast.

9. The method for inducing an immune response in an animal according to claim 8 wherein said yeast is Saccharomyces cerevisiae.

10. The method for inducing an immune response in an animal according to claim 8 wherein said antigen is derived from a virus.

11. A method for inducing an immune response in an animal comprising:
providing an oral formulation of transformed Saccharomyces cerevisiae wherein said transformed Saccharomyces cerevisiae comprises a heterologous nucleic acid encoding for an immunoprotective epitope from influenza A.

12. A method for inducing an immune response in an animal according to claim 11 wherein said immunoprotective epitope is influenza HA or NA.

13. An immunogenic composition comprising:
an oral formulation of a microflora organism having an expression vector wherein said expression vector comprises a heteroligous nucleic acid that encodes for an antigen.

14. The immunogenic composition comprising according to claim 13 wherein said microflora organism is a yeast or bacteria.

15. The immunogenic composition comprising according to claim 13 wherein said antigen is selected from the group consisting of tumors, bacteria, viruses, parasites, and fungi.

16. The immunogenic composition comprising according to claim 15 wherein said viruses are selected from the group consisting of influenza, hepatitis, HIV, and rotavirus.

17. The immunogenic composition comprising according to claim 14 wherein said yeast in is selected from the group consisting of Saccharomyces cerevisiae, S. exiquus, S. telluris, S. dairensis., S. servazzii, S. unisporus, and S. kluyveri.

18. The immunogenic composition comprising according to claim 14 wherein said bacteria in is selected from the group consisting of Bifidobacterium sp, Streptococcus thermophilus, Enterococcus faecalis, Enterococcus durans, Lactococcus lactis, Lactobacillus lactis, Lactobacillus acidophilus, Lactobacillus bulgaricus, Lactobacillus thermophilus, Lactobacillus casei and Lactobacillus plantarum.

19. The immunogenic composition comprising according to claim 13 wherein said oral formulation is selected from the group consisting of powder, a freeze dried powder a liquid preparation, a semi-solid, yogurt milk and cheese.

20. An immunogenic composition comprising:
an oral formulation of transformed yeast wherein said yeast comprise a heterologous nucleic acid encoding for an antigen where in said antigen is expressed on the surface of said yeast.

21. The immunogenic composition comprising according to claim 20 wherein said yeast is Saccharomyces cerevisiae.

22. The immunogenic composition comprising according to claim 20 wherein said antigen is derived from a virus.

23. An immunogenic composition comprising:
an oral formulation of transformed Saccharomyces cerevisiae wherein said transformed Saccharomyces cerevisiae comprises a heterologous nucleic acid encoding for an immunoprotective epitope from influenze A.

24. The immunogenic composition comprising according to claim 23 wherein said immunoprotective epitope is inflenza HA or NA.
